(19) United States
(12) Patent Application Publication
(10) Pub. No.: US 2024/0207452 A1
(43) Pub. Date: Jun. 27, 2024

(54) NOVEL COMPOSITIONS WITH BRAIN-SPECIFIC TARGETING MOTIFS AND COMPOSITIONS CONTAINING SAME

(71) Applicant: The Trustees of the University of Pennsylvania, Philadelphia, PA (US)

(72) Inventors: Joshua Joyner SIMS, Villanova, PA (US); James M. WILSON, Philadelphia, PA (US); Yuan YUAN, Boston, MA (US)

(73) Assignee: The Trustees of the University of Pennsylvania, Philadelphia, PA (US)

(21) Appl. No.: 18/556,003

(22) PCT Filed: Apr. 22, 2022
(86) PCT No.: PCT/US2022/025879

§ 371 Date: Oct. 18, 2023

Related U.S. Application Data
(60) Provisional application No. 63/178,881, filed on Apr. 23, 2021.

(51) Int. Cl. A61K 48/00; C07K 14/005; C07K 14/46; C12N 15/86

(52) U.S. Cl. CPC A61K 48/0058; C07K 14/005; C07K 14/46; C12N 15/86; C12N 2750/14122; C12N 2750/14143; C12N 2750/14145

(57) ABSTRACT

Provided herein are compositions including brain-capillary binding and/or blood-brain barrier crossing (BBB) tissue-targeting peptides linked thereto or inserted in a targeting protein of a recombinant vector having at least one exogenous peptide comprising an amino acid sequence of Y-G/A/R/K-Y/H-GNPA-T/R/H-RYFD-V/K. Compositions providing such conjugates, targeting peptides, or recombinant vectors having a mutant capsid or envelope protein are provided as are uses thereof.

### BACKGROUND OF THE INVENTION

The Adeno-Associated Virus (AAV) is currently the gene therapy vector of choice. AAVs can deliver a transgene that is stably expressed long-term from a non-integrating genome, and because the AAV is not associated with any human diseases. However, AAV gene therapy is currently limited to a small number of diseases due to challenges in delivery and tropism. This is particularly true for disorders of the central nervous system (CNS). Direct delivery of AAV gene therapy vectors is possible, by injecting the vector directly into the cerebrospinal fluid (CSF), but this method typically transduces 1% or less of brain cells. Furthermore, most of that transduction is concentrated on the cells that are in direct contact with the CSF. Cells in the “deep brain” are rarely transduced. This has limited the number of CNS disorders treatable by gene therapy.

In contrast to the CSF network, the vascular system of the brain reaches nearly every cell in the CNS. This is because of a high demand these tissues have for glucose, oxygen, and other nutrients. However, cells in the brain and spinal cord are protected from the circulatory system by a specialized vascular unit, the Blood Brain Barrier (BBB). The BBB limits the diffusion of large molecules like viral vectors and proteins, and even many small molecule drugs through a complex network of tightly linked cells that surround the blood vessels of the brain and spinal cord. Thus, a grand challenge in gene therapy delivery to the CNS has been the engineering of an AAV variant capable of crossing the BBB at high efficiency and transducing cells in the deep brain.

One AAV capsid developed at the California Institute of Technology (CalTech) has a seven amino acid peptide inserted into hypervariable loop 8 (HVR8) on the AAV9 capsid to generate a rAAV called AAV9-PHP.B which is reported to mediate interaction with Ly6a, a GPI-anchored receptor on the brain vasculature of some mouse strains. US Patent Published Application No. 2017/0166926A1. This interaction drives transport of AAV9-PHP.B across the BBB, resulting in ˜50-fold higher transduction of brain cells than AAV9. However, this finding has not translated to larger animals or humans.

There remains a need for vectors which can specifically target selected tissue and cell types.

### SUMMARY OF THE INVENTION

In certain embodiments, a recombinant adeno-associated particle (rAAV) having capsid comprising an amino acid sequence that comprises an exogenous targeting peptide inserted into the hypervariable region, wherein the exogenous targeting peptide comprises the optional N-terminal linker—Y-X-X″-GNPA-X″″-RYFD-X″″ (SEQ ID NO: 14)—optional C-terminal linker is provided, wherein X′ is G, A, R, or K, X″ is Y or H, X″″ is T, R or H, and X″ is V or K (e.g., YGY-GNPA-T/R/H-RYFD-V/K or YAY-GNPA-T/R/H-RYFD-V/K). Suitably, the amino acid sequence is part of at least the AAV VP3 protein in the capsid and a vector genome packaged in the capsid which comprises a nucleic acid sequence encoding a gene product under control of sequences which direct expression thereof. In certain embodiments, the exogenous targeting peptide is inserted in the hypervariable region VIII or IV of a parental capsid. In some embodiments, the parental capsid is selected from AAV9, AAV8, AAV7, AAV6, AAV5, AAV4, AAV3, AAV1, AAVhu68, and AAVrh.91. In some embodiments, the suitable location for insertion is a hypervariable region VIII. In further embodiments, the suitable location for insertion is between amino acids 588 and 589 as determined based on the numbering of VP1 amino acid sequence of SEQ ID NO: 9 (AAV9).

In certain embodiments, the exogenous targeting peptide sequence inserted into the capsid comprises Y-X′-X″-GNPA-X′″-RYFD-X″ peptide motif (SEQ ID NO: 14) wherein X′ is G, A, R, or K, X″ is Y or H, X′″ is T, R or H, and X″ is V or K (also referred to as YGY-GNPA-T/R/H-RYFD-V/K). In some embodiments, the exogenous targeting peptide comprises: (a) YGYGNPATRYFDV (SEQ ID NO: 1); (b) YGYGNPARRYFDV (SEQ ID NO: 6); (c) YGYGNPAHRYFDV (SEQ ID NO: 7); or (d) YGYGNPATRYFDK (SEQ ID NO: 8). In other embodiments, exogenous targeting peptide comprises: (a) YAYGNPATRYFDV (SEQ ID NO: 2); (b) YKYGNPATRYFDV (SEQ ID NO: 3); (c) YRYGNPATRYFDV (SEQ ID NO: 4); or (d) YGHGNPATRYFDV (SEQ ID NO: 5).

In certain embodiments, a composition comprises the rAAV having the exogenous targeting peptide and optional flanking sequences and one or more of a physiologically compatible carrier, excipient, and/or aqueous suspension base.

In certain embodiments, a recombinant brain cell-targeting peptide is provided, the peptide comprising Y-X-X″-GNPA-X″″-RYFD-X″″ peptide motif (SEQ ID NO: 14) wherein X′ is G, A, R, or K, X″ is Y or H, X″ is T, R or H, and X″ is V or K (also referred to as Y-G/A/R/K-Y/H-GNPA-T/R/H-RYFD-V/K), optionally flanked at the amino terminus and/or the carboxy terminus of SEQ ID NO: 14 by two amino acids to seven amino acids, and optionally the peptide or peptide with linker(s) are conjugated to a nanoparticle, a second molecule, or a recombinant viral capsid protein. In certain embodiments, the brain cell-targeting peptide comprises: (a) YGYGNPATRYFDV (SEQ ID NO: 1): (b) YAYGNPATRYFDV (SEQ ID NO: 2); (c) YKYGNPATRYFDV (SEQ ID NO: 3); (d) YRYGNPATRYFDV (SEQ ID NO: 4); (e) YGHGNPATRYFDV (SEQ ID NO: 5); (f) YGYGNPARRYFDV (SEQ ID NO: 6); (g) YGYGNPAHRYFDV (SEQ ID NO: 7); or (h) YGYGNPATRYFDK (SEQ ID NO: 8). In some embodiments, the brain cell-targeting peptide is YGYGNPATRYFDV. In other embodiments, the brain cell-targeting peptide is YAYGNPATRYFDV. In certain embodiments, a composition is provided which comprises the endothelial cell targeting peptide and one or more of a physiologically compatible carrier, excipient, and/or aqueous suspension base.

In certain embodiments, a fusion polypeptide or protein comprising a brain cell-targeting peptide and a fusion partner which comprises at least one polypeptide or protein is provided herein. In certain embodiments, a composition comprising a fusion polypeptide or protein as provided herein and one or more of a physiologically compatible carrier, excipient, and/or aqueous suspension base.

Provided herein are compositions and methods for using an rAAV, an endothelial cell targeting peptide, a fusion polypeptide or protein, and/or a composition as described herein of for delivering a therapeutic to a patient in need thereof. In certain embodiments, the therapeutic is targeted to the brain endothelial cells.

In certain embodiments, a composition and/or method is provided for treating Allan-Herndon-Dudley disease by delivering to a subject in need thereof an rAAV as described herein wherein the encoded gene product is an MCT8 protein.

In certain embodiments, a method is provided for targeting therapy to the brain, comprising administering to a patient in need thereof an rAAV as described herein. In certain embodiments, a method is provided for treating one or more of cognitive, neurodegenerative, and/or disease of the central nervous system by delivering to a subject in need thereof a stock of the rAAV as described herein. In some embodiments, a method is provided of treating of various infections of the central nervous system.

In certain embodiments, a method is provided for increasing transduction of AAV production cells in vitro comprising inserting a Y-X′-X″-GNPA-X′″-RYFD-X″ peptide motif (SEQ ID NO: 14) wherein X′ is G, A, R, or K, X″ is Y or H, X″″ is T, R or H, and X″ is V or K (also referred to as Y-G/A/R/K-Y/H-GNPA-T/R/H-RYFD-V/K) peptide core motif into an AAV capsid. In certain embodiments, the production cells are 293 cells.

These and other embodiments and advantages of the invention will be apparent from the specification, including, without limitation, the detailed description of the invention.

### BRIEF DESCRIPTION OF THE FIGURES

FIGS. 1A and 1B show enrichment for the top performing peptide hits in the mouse (C57BL/6J and Balb/c) brain. FIG. 1A shows enrichment for the top performing peptide hits in brain from primary screen in C57BL/6J mice. FIG. 1B shows enrichment for the top performing peptide hits in brain from primary screen in Balb/c mice.

FIGS. 2A and 2B show yield and BBB scores from screening of “YGY”-based variant libraries. FIG. 2A shows yield and BBB score from screening of a library with truncation-based “YGY” variants. FIG. 2B shows yield and BBB score from screening of a library with single point mutant “YGY” variants.

FIGS. 3A and 3B show results of functional studies of AAV9-YGY-Variants. FIG. 3A shows yield of a small and medium scale productions of the top 7 performing inserts in AAV9-variant capsids comprising GFP transgene, and compared with yields of AAV9 and AAV9-variant comprising original YGY peptide insert. FIG. 3B shows a preliminary transduction test with GFP vectors in mouse (C57BL/6J) brain plotted as a ratio of mRNA copy number over micro-gram total mRNA.

FIGS. 4A and 4B show AAV9 or AAV9 variant biodistribution in mice. FIG. 4A shows AAV9 or AAV9 variant biodistribution in mouse (C57BL/6J) brain, plotted as GC/μg DNA. FIG. 4B shows AAV9 or AAV9 variant biodistribution in mouse (C57BL/6J) liver, plotted as GC/μg DNA.

FIGS. 5A and 5B show results of the transduction test with AAV-GFP vectors in mice. FIG. 5A shows a transduction test with GFP vectors in mouse (Balb/c) brain plotted as a ratio of mRNA copy number over micro-gram total mRNA. FIG. 5B shows a transduction test with GFP vectors in mouse liver plotted as a ratio of mRNA copy number over micro-gram total mRNA.

FIGS. 6A and 6B show the enrichment scores for the top performing peptide hits in mouse brain in the screen, with referenced peptides. FIG. 6A shows enrichment scores for C57BL/6J mice. FIG. 6B shows enrichment scores for Balb/c mice.

FIG. 7 shows sample production yield (plotted as GC/cell stack) for AAV9-YGY and AAV9-YGY2A.

FIGS. 8A and 8B show the enrichment scores for the top performing hits in NHP tissue in the screen. FIG. 8A shows enrichment scores for NHP brain. FIG. 8B shows enrichment scores for NHP spinal cord tissue.

FIGS. 9A and 9B show a secondary validation of the transduction levels of top performing peptide hits in AAV capsid comprising GFP reporter transgene in Balb/c mice. The results are plotted relative to AAV9 transduction. FIG. 9A shows secondary validation screen of selected peptide hits targeting of brain tissue in Balb/c mice. FIG. 9B shows secondary validation screen of selected peptide hits targeting of liver tissue in Balb/c mice.

FIGS. 10A to 10C show a secondary validation of the transduction levels of top performing peptide hits in AAV capsid comprising GFP reporter transgene in C57BL/6J mice. The results are plotted relative to AAV9 transduction. FIG. 10A shows secondary validation screen of selected peptide hits targeting of brain tissue in C57BL/6J mice (DNA). FIG. 10B shows secondary validation screen of selected peptide hits targeting of brain tissue in C57BL/6J mice (RNA). FIG. 10C shows secondary validation screen of selected peptides hits targeting of liver tissue in C57BL/6J mice.

FIGS. 11A to 11H show biodistribution in various tissues (plotted as Copies/μg gDNA (qPCR)) of AAV2/9-YGY and AAV2/9-YGY2A vectors in C57BL/6J mice, compared to AAV9 vectors. FIG. 11A shows biodistribution in brain (plotted as Copies/μg gDNA (qPCR)) of AAV2/9-YGY and AAV2/9-YGY2A vectors in C57BL/6J mice, compared to AAV9 vectors. FIG. 11B shows biodistribution in heart (plotted as Copies/μg gDNA (qPCR)) of AAV2/9-YGY and AAV2/9-YGY2A vectors in C57BL/6J mice, compared to AAV9 vectors. FIG. 11C shows biodistribution in liver (plotted as Copies/μg gDNA (qPCR)) of AAV2/9-YGY and AAV2/9-YGY2A vectors in C57BL/6J mice, compared to AAV9 vectors. FIG. 11D shows biodistribution in muscle (plotted as Copies/μg gDNA (qPCR)) of AAV2/9-YGY and AAV2/9-YGY2A vectors in C57BL/6J mice, compared to AAV9 vectors. FIG. 11E shows biodistribution in spinal cord (plotted as Copies/μg gDNA (qPCR)) of AAV2/9-YGY and AAV2/9-YGY2A vectors in C57BL/6J mice, compared to AAV9 vectors. FIG. 11F shows biodistribution in lung (plotted as Copies/μg gDNA (qPCR)) of AAV2/9-YGY and AAV2/9-YGY2A vectors in C57BL/6J mice, compared to AAV9 vectors. FIG. 11G shows biodistribution in kidney (plotted as Copies/μg gDNA (qPCR)) of AAV2/9-YGY and AAV2/9-YGY2A vectors in C57BL/6J mice, compared to AAV9 vectors. FIG. 11H shows biodistribution in spleen (plotted as Copies/μg gDNA (qPCR)) of AAV2/9-YGY and AAV2/9-YGY2A vectors in C57BL/6J mice, compared to AAV9 vectors.

FIGS. 12A to 12H show biodistribution in various tissues (plotted as Copies Relative to AAV2/9) of AAV2/9-YGY and AAV2/9-YGY2A vectors in C57BL/6J mice, compared to AAV9 vectors. FIG. 12A shows biodistribution in brain (plotted as Copies Relative to AAV2/9) of AAV2/9-YGY and AAV2/9-YGY2A vectors in C57BL/6J mice, compared to AAV9 vectors. FIG. 12B shows biodistribution in heart (plotted as Copies Relative to AAV2/9) of AAV2/9-YGY and AAV2/9-YGY2A vectors in C57BL/6J mice, compared to AAV9 vectors. FIG. 12C shows biodistribution in liver (plotted as Copies Relative to AAV2/9) of AAV2/9-YGY and AAV2/9-YGY2A vectors in C57BL/6J mice, compared to AAV9 vectors. FIG. 12D shows biodistribution in muscle (plotted as Copies Relative to AAV2/9) of AAV2/9-YGY and AAV2/9-YGY2A vectors in C57BL/6J mice, compared to AAV9 vectors. FIG. 12E shows biodistribution in spinal cord (plotted as Copies Relative to AAV2/9) of AAV2/9-YGY and AAV2/9-YGY2A vectors in C57BL/6J mice, compared to AAV9 vectors. FIG. 12F shows biodistribution in lung (plotted as Copies Relative to AAV2/9) of AAV2/9-YGY and AAV2/9-YGY2A vectors in C57BL/6J mice, compared to AAV9 vectors. FIG. 12G shows biodistribution in kidney (plotted as Copies Relative to AAV2/9) of AAV2/9-YGY and AAV2/9-YGY2A vectors in C57BL/6J mice, compared to AAV9 vectors. FIG. 12H shows biodistribution in spleen (plotted as Copies Relative to AAV2/9) of AAV2/9-YGY and AAV2/9-YGY2A vectors in C57BL/6J mice, compared to AAV9 vectors.

FIG. 13 shows an alignment of the specified region of the amino acid sequences of the various AAV capsid proteins of AA9 (amino acids 566 to 615 of AAV9 capsid; SEQ ID NO: 30), AAV8 (amino acids 565 to 614 of AAV8 capsid; SEQ ID NO: 31), AAV7 (amino acids 567 to 616 of AAV7; SEQ ID NO: 32), AAV6 (amino acids 550 to 599 of AAV6 capsid; SEQ ID NO: 33), AAV5 (amino acids 556 to 605 of AAV5; SEQ ID NO: 34), AAV4 (amino acids 558 to 607 of AAV4 capsid; SEQ ID NO: 35), AAV3B (amino acids 564 to 613 of AAV3B capsid; SEQ ID NO: 36), AAV2 (amino acids 566 to 615 of AAV2 capsid; SEQ ID NO: 37), and AAV1 (amino acids 566 to 615 of AAV1 capsid; SEQ ID NO: 38), which is focused on the region HVRVIII in which the targeting peptide may be inserted (based on structure analysis).

### DETAILED DESCRIPTION OF THE INVENTION

In certain embodiments, a targeting peptide and nucleic acid sequences encoding same are provided herein. Also provided herein are fusion proteins, modified proteins, mutant viral capsids and other moieties operably linked to an exogenous targeting peptide motif Y-G/A/R/K-Y/H-GNPA-T/R/H-RYFD-V/K motif. With reference to SEQ ID NO: 14, the motif may be represented as Y-X′-X″-GNPA-X″-RYFD-X″ motif (SEQ ID NO: 14), wherein X′ is G, A, R, K, X″ is Y or H, X″ is T, R or H, and X″ is V or K. In certain embodiments, X″ is Y and X′ is G (YGY) or A (YAY, also termed YGY2A). Also provided herein are nucleic acid sequences encoding same. In certain embodiments, this exogenous motif modifies the native tissue specificity of the source (parental) protein, viral vector, or other moiety. In certain embodiments, compositions having one or more of these targeting peptides have enhanced or altered Blood Brain Barrier (BBB) targeting. In certain embodiments, compositions having one or more of these targeting peptides have enhanced or altered brain capillary targeting. In certain embodiments, viral vectors having modified capsids containing this motif exhibit increased transduction of AAV production cells in vitro.

Advantageously, in certain embodiments, mutant rAAV capsids comprising the Y-X′-X″-GNPA-X″-RYFD-X″ motif (SEQ ID NO: 14), wherein X′ is G, A, R, K, X″ is Y or H, X″ is T, R or H, and X″ is V or K (SEQ ID NO: 14), targeting peptide provided herein provide significant transduction advantages in the central nervous system, including the brain and/or the spinal cord as compared to a parental capsid (e.g., AAV9 or a another clade F capsid). In certain embodiments, mutant rAAV capsids comprising the Y-X′-X″-GNPA-X″-RYFD-X″ motif (SEQ ID NO: 14), wherein X′ is G, A, R, K, X″ is Y or H, X″ is T, R or H, and X″ is V or K (SEQ ID NO: 14), more specifically wherein the X′ is G or A, targeting peptide provide significant transduction advantages in the central nervous system, including the brain and/or the spinal cord, as compared to a parental capsid. In certain embodiments, mutant rAAV capsids comprising the targeting peptides, as provided herein, demonstrate reduced transduction (i.e., de-targeted/ing) to heart, lung, liver and/or kidney as compared to its parental capsid (e.g., AAV9 or another clade F capsid or other modification thereof (e.g., AAV9-PHP.B)). In certain embodiments, these targeting peptides provide similar function to other mutant viral vector or non-viral constructs, as described herein.

The targeting peptide may be linked to a recombinant protein (e.g., for enzyme replacement therapy) or polypeptide (e.g., an immunoglobulin) to form a fusion protein or a conjugate to target a desired tissue (e.g., CNS). Additionally, the targeting peptide may be linked to a liposome and/or a nanoparticle (a lipid nanoparticle, LNP) forming a peptide-coated liposome and/or LNP to target the desired tissue. Sequences encoding at least one copy of a targeting peptide and optional linking sequences may be fused in frame with the coding sequence for the recombinant protein and co-expressed with the protein or polypeptide to provide fusion proteins or conjugates. Alternatively, other synthetic methods may be used to form a conjugate with a protein, polypeptide or another moiety (e.g., DNA, RNA, or a small molecule). In certain embodiments, multiple copies of a targeting peptide are in the fusion protein/conjugate. Suitable methods for conjugating a targeting peptide to a recombinant protein include modifying the amino (N)-terminus and one or more residues on a recombinant human protein (e.g., an enzyme) using a first crosslinking agent to give rise to a first crosslinking agent modified recombinant human protein, modifying the amino (N)-terminus of a short extension linker region preceding a targeting peptide using a second crosslinking agent to give rise to a second crosslinking agent modified variant target peptide, and then conjugating the first crosslinking agent modified recombinant human protein to the second crosslinking agent modified variant targeting peptide containing a short extension linker. Other suitable methods for conjugating a targeting peptide to a recombinant protein include conjugating a first crosslinking agent modified recombinant human protein to one or more second crosslinking agent modified variant targeting peptides, wherein the first crosslinking agent modified recombinant protein comprises a recombinant protein characterized as having a chemically modified N-terminus and one or more modified lysine residues and the one or more second crosslinking agent modified variant targeting peptides comprise one or more variant targeting peptides comprising a modified N-terminal amino acid of a short extension linker preceding the targeting peptide. Still other suitable methods for conjugating a targeting peptide to a protein, polypeptide, nanoparticle, or another biologically useful chemical moiety may be selected. See, e.g., U.S. Pat. No. 9,545,450 B2 (NHS-phosphine cross-linking agents: NHS-Azide cross-linking agents); US Published Patent Application No. US 2018/0185503 A1 (aldehyde-hydrazide crosslinking).

In certain embodiments, the targeting peptide may be inserted into a suitable site within a protein or polypeptide (e.g., a viral capsid protein). In certain embodiments, a targeting peptide may be flanked at its carboxy (COO—) and/or amino (N—) terminus by a short extension linker. Such a linker may be about 1 to about 20 amino acid residues in length, or about 2 to about 20 amino acids residues, or about 1 to about 15 amino acid residues, or about 2 to about 12 amino acid residues, or about 2 to about 7 amino acid residues in length. The short extension linker can also be about 10 amino acids in length. The presence and length of a linker at the N-terminus is independently selected from a linker at the carboxy-terminus, and the presence and length of a linker at the carboxy terminus is independently selected from a linker at the N-terminus. Suitable short extension linkers can be provided using an about 5-amino acid flexible GS extension linker (glycine-glycine-glycine-glycine-serine), an about 10-amino acid extension linker comprising 2 flexible GS linkers, an about 15-amino acid extension linker comprising 3 flexible GS linkers, an about 20-amino acid extension linker comprising 4 flexible GS linkers, or any combination thereof.

In certain embodiment, a composition is provided which is useful for targeting a brain cell. The composition is a mutant capsid, fusion protein or another conjugate comprising at least one exogenous targeting peptide comprising: a core amino acid sequence of YGYGNPATRYFDV (SEQ ID NO: 1) optionally flanked at its amino terminus and/or carboxy terminus of the core sequence by two amino acids to seven amino acids, and optionally further conjugated to a nanoparticle, a second molecule, or a viral capsid protein. In certain embodiments, the composition is a mutant capsid, fusion protein or another conjugate comprising at least one exogenous targeting peptide comprising: a core amino acid sequence of YAYGNPATRYFDV (SEQ ID NO: 2) optionally flanked at its amino terminus and/or carboxy terminus of the core sequence by two amino acids to seven amino acids, and optionally further conjugated to a nanoparticle, a second molecule, or a viral capsid protein. The targeting peptide comprising the following core amino acid sequences with optional linking sequences:

(SEQ ID NO: 2)
(a) YAYGNPATRYFDV;
(SEQ ID NO: 3)
(b) YKYGNPATRYFDV;
(SEQ ID NO: 4)
(c) YRYGNPATRYFDV;
(SEQ ID NO: 5)
(d) YGHGNPATRYFDV;
(SEQ ID NO: 6)
(e) YGYGNPARRYFDV;
(SEQ ID NO: 7)
(f) YGYGNPAHRYFDV;
or
(SEQ ID NO: 8)
(g) YGYGNPATRYFDK.

In certain embodiments, the targeting peptide core amino acid sequence is encoded by a nucleic acid sequence selected from:
(a) tacggctacg gcaacccege caccegctac ttcgacgtg (SEQ ID NO: 25); or
(b) tatgcgtatg gcaacccggc gacccgttat tttgatgtg (SEQ ID NO: 24).

In certain embodiments, the targeting peptide core amino acid sequence is encoded by a nucleic acid sequence of SEQ ID NO: 25, or a sequence at least about 70% identical thereto. In certain embodiments, the targeting peptide core is encoded by a nucleic acid sequence of SEQ ID NO: 24, or a sequence at least about 70% identical thereto. In some embodiments, the nucleic acid sequence encoding for the targeting peptide core is optionally flanked at the 5′ and/or 3′ ends of the nucleic acid sequence of the core peptide sequence by six to twenty-one nucleotides of an extension linker.

In certain embodiments, the targeting peptide core is YGYGNPATRYFDV (SEQ ID NO: 1). In certain embodiments, the targeting peptide core is YAYGNPATRYFDV (SEQ ID NO: 2). In certain embodiments, the targeting peptide core is YKYGNPATRYFDV (SEQ ID NO: 3). In certain embodiments, the targeting peptide core is YRYGNPATRYFDV (SEQ ID NO: 4). In certain embodiments, the targeting peptide core is YGHGNPATRYFDV (SEQ ID NO: 5). In certain embodiments, the targeting peptide core is YGYGNPARRYFDV (SEQ ID NO: 6). In certain embodiments, the targeting peptide core is YGYGNPAHRYFDV (SEQ ID NO: 7). In certain embodiments, the targeting peptide core is YGYGNPATRYFDK (SEQ ID NO: 8). In certain embodiment, more than one copy of a targeting peptide within this motif is provided in a conjugate or modified protein (e.g., a parvovirus capsid). In certain embodiments, two or more different targeting peptide cores are present.

In certain embodiment, a composition is provided which is useful for targeting a brain cell and/or cell that is in direct contact with cerebrospinal fluid (CSF). In certain embodiment, a composition is provided which is useful for targeting a cell in deep brain. In certain embodiment, a composition is provided which is useful for targeting cells in spinal cord. In certain embodiment, a composition is provided which is useful for targeting cells in brain and/or spinal cord, while also de-targeting cells in heart, lung, liver and/or kidney tissues. In certain embodiment, a composition is provided which is useful for targeting cells in brain and/or spinal cord, while also de-targeting cells in liver. The composition is a mutant capsid, fusion protein or another conjugate comprising at least one exogenous targeting peptide comprising: an amino acid core sequence of Y-X″-X″-GNPA-X″-RYFD-X″ motif (SEQ ID NO: 14), wherein X′ is G, A, R, K, X″ is Y or H, X″ is T, R or H, and X″ is V or K, wherein the targeting peptide optionally flanked at the amino terminus and/or the carboxy terminus of the motif by two amino acids to seven amino acids, and optionally further conjugated to a nanoparticle, a second molecule, or a viral capsid protein. In certain embodiments, composition is a mutant capsid, fusion protein or another conjugate comprising at least one exogenous targeting peptide comprising: an amino acid core sequence of YGYGNPATRYFDV (SEQ ID NO: 1) optionally flanked at the amino terminus and/or the carboxy terminus of the motif by two amino acids to seven amino acids, and optionally further conjugated to a nanoparticle, a second molecule, or a viral capsid protein. In certain embodiments, composition is a mutant capsid, fusion protein or another conjugate comprising at least one exogenous targeting peptide comprising: an amino acid core sequence of YAYGNPATRYFDV (SEQ ID NO: 2) optionally flanked at the amino terminus and/or the carboxy terminus of the motif by two amino acids to seven amino acids, and optionally further conjugated to a nanoparticle, a second molecule, or a viral capsid protein. The targeting peptide comprises: a core sequence of (a) YAYGNPATRYFDV (SEQ ID NO: 2); (b) YKYGNPATRYFDV (SEQ ID NO: 3); (c) YRYGNPATRYFDV (SEQ ID NO: 4); (d) YGHGNPATRYFDV (SEQ ID NO: 5); (e) YGYGNPARRYFDV (SEQ ID NO: 6); (f) YGYGNPAHRYFDV (SEQ ID NO: 7); or (g) YGYGNPATRYFDK (SEQ ID NO: 8).

Examples of suitable proteins, including enzymes, immunoglobulins, therapeutic proteins, immunogenic polypeptides, nanoparticles, DNA, RNA, and other moieties (e.g., small molecules, etc.) for targeting are described in more detail below. These and other biologic and chemical moieties are suitable for use with the targeting peptide(s) provided herein.

In certain embodiments, a composition is a nucleic acid sequence molecule, wherein the nucleic acid sequence is a DNA molecule or RNA molecule, e.g., naked DNA, naked plasmid DNA, messenger RNA (mRNA), containing the targeting peptide sequence motif linked to the nucleic acid molecule. In some embodiments, the nucleic acid molecule is further coupled with various compositions and nano particles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., WO2014/089486, US 2018/0353616A1, US2013/0037977A1, WO2015/074085A1, U.S. Pat. No. 9,670,152B2, and U.S. Pat. No. 8,853,377B2, X. Su, et al., Mol. Pharmaceutics, 2011, 8 (3), pp 774-787; web publication: Mar. 21, 2011; WO2013/182683, WO 2010/053572 and WO 2012/170930, all of which are incorporated herein by reference. In certain embodiments, the targeting peptide motif is chemically linked to a nanoparticle surface, wherein the nanoparticle encapsulates a nucleic acid molecule. In some embodiments the nanoparticle comprising the targeting peptide linked to the surface is designed for targeted tissue-specific delivery. In some embodiments two or more different targeting peptides are linked to the surface of the nanoparticle. Suitable chemical linking or cross-linking include those known to one skilled in the art.

### Capsids

In certain embodiments, a recombinant parvovirus is provided which has a modified parvovirus capsid having at least exogenous peptide from the Y-G/A/R/K-Y/H-GNPA-T/R/H-RYFD-V/K (SEQ ID NO: 14) core targeting motif. The motif in SEQ ID NO: 14 may be represented as Y-X′-X″-GNPA-X″-RYFD-X″ motif, wherein X′ is G, A, R, K, X″ is Y or H, X′″ is T, R or H, and X″ is V or K. Such a recombinant parvovirus may be a hybrid bocavirus/AAV or a recombinant AAV vector (rAAV). In other embodiments, other viral vectors may be generated having one or more exogenous targeting peptides from the Y-G/A/R/K-Y/H-GNPA-T/R/H-RYFD-V/K (SEQ ID NO: 14) core targeting motif (which may be same or different, or combinations thereof) in an exposed capsid protein to modulate and/or alter the targeting specificity of the viral vector as compared to the parental vector.

The targeting peptide may be inserted into a hypervariable loop (HVR) VIII (also referenced as HVR8) at any suitable location. For example, based on the numbering of the AAV9 capsid, the peptide is inserted with linkers of various lengths between amino acids 588 and 589 (Q-A) of the AAV9 capsid protein, based on the numbering of the AAV9 VP1 (also referenced as Vp1 or vp1) amino acid sequence; SEQ ID NO: 9. See, also, WO 2019/168961, published Sep. 6, 2019, including Table G providing the deamidation pattern for AAV9 and WO 2020/160582, filed Sep. 7, 2018. The amino acid residue locations (i.e., amino acid numbering reference) are identical in AAVhu68 (SEQ ID NO: 10). However, another site may be selected within HVRVIII. Alternatively, another exposed loop HVR (e.g., HVRIV) may be selected for the insertion. Comparable HVR regions may be selected in other capsids. In certain embodiments, the location for the HVRVIII and HVRIV is determined using an algorithm and/or alignment technique as described in U.S. Pat. No. 9,737,618 B2 (column 15, lines 3-23), and U.S. Pat. No. 10,308,958 B2 (column 15, line 46-column 16, line 6), which are incorporated herein by reference in its entirety. In certain embodiments, the targeting peptide may be inserted into a hypervariable loop HVRVIII as described in U.S. Provisional Patent Application No. 63/119,863, filed Dec. 1, 2020, and International Patent Application No. PCT/US2021/061312, filed Dec. 1, 2022, which are incorporated herein by reference in their entireties. In certain embodiments, AAV1 capsid protein is selected as a parental capsid, wherein the targeting peptide with linkers of various lengths is inserted in a suitable location of the HVRVIII region of amino acid 582 to 585, or HVRIV region of amino acid 456 to 459 based on vp1 numbering (Gurda, B L., et al., Capsid Antibodies to Different Adeno-Associated Virus Serotypes Bind Common Regions, 2012, Journal of Virology, Jun. 12, 2013, 87(16): 9111-91114). In certain embodiments, AAV8 is selected as parental capsid, wherein the targeting peptide with linkers of various length is inserted in a suitable location of HVRVIII region of amino acid 586 to 591, or HVRIV region of amino acid 456 to 460, based on VP1 numbering (Gurda, B L., et al., Mapping a Neutralizing epitope onto the Capsid of Adeno-Associated Virus Serotype 8, 2012, Journal of Virology, May 16, 2012, 86(15): 7739-7751).

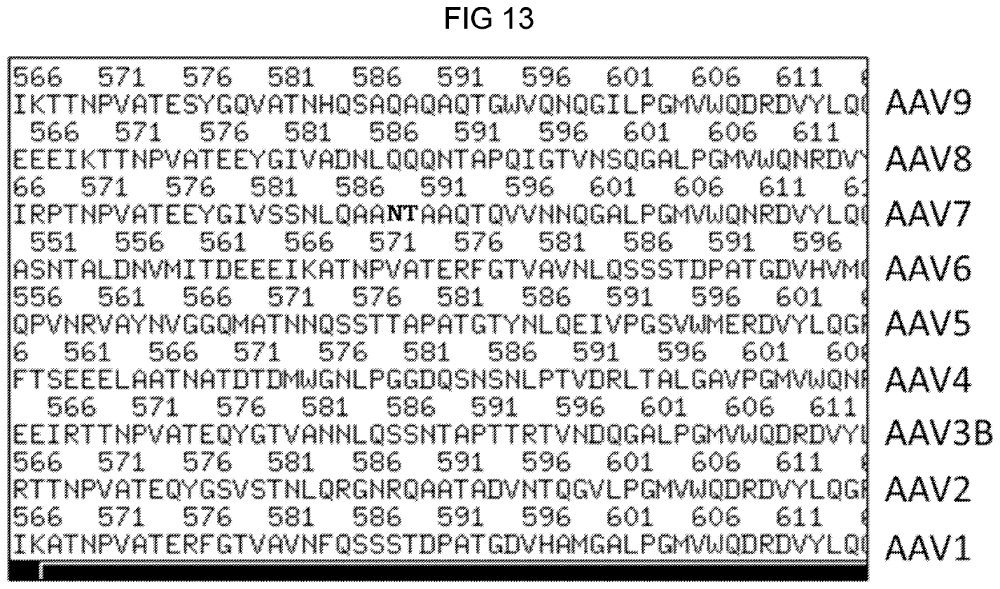

In certain embodiments, AAV9 is selected as parental capsid, wherein the targeting peptide with linkers of various length is inserted in a suitable location of HVRVIII region of amino acid 588 and 589 (Q-A), based on VP1 numbering. In other embodiments, AAVhu68 or another clade F capsid is selected as the parental capsid. In certain embodiments, AAV8 is selected as parental capsid, wherein the targeting peptide with linkers of various length is inserted in a suitable location of HVRVIII region of amino acid 590 and 591 (N-T), based on VP1 numbering. In certain embodiments, AAV7 is selected as parental capsid, wherein the targeting peptide with linkers of various length is inserted in a suitable location of HVRVIII region of amino acid 589 and 590 (N-T), based on VP1 numbering. In certain embodiments, AAV6 is selected as parental capsid, wherein the targeting peptide with linkers of various length is inserted in a suitable location of HVRVIII region of amino acid 588 and 589 (S-T), based on VP1 numbering. In certain embodiments, AAV5 is selected as parental capsid, wherein the targeting peptide with linkers of various length is inserted in a suitable location of HVRVIII region of amino acid 577 and 578 (T-T), based on VP1 numbering. In certain embodiments, AAV4 is selected as parental capsid, wherein the targeting peptide with linkers of various length is inserted in a suitable location of HVRVIII region of amino acid 586 and 587 (S-N), based on VP1 numbering. In certain embodiments, AAV3/3B is selected as parental capsid, wherein the targeting peptide with linkers of various length is inserted in a suitable location of HVRVIII region of amino acid 588 and 589 (N-T), based on VP1 numbering. In certain embodiments, AAV2 is selected as parental capsid, wherein the targeting peptide with linkers of various length is inserted in a suitable location of HVRVIII region of amino acid 587 and 589 (N-R), based on VP1 numbering. In certain embodiments, AAV1 is selected as parental capsid, wherein the targeting peptide with linkers of various length is inserted in a suitable location of HVRVIII region of amino acid 588 and 589 (S-T), based on VP1 numbering. See also, FIG. 13 which shows an alignment of the specified region of the amino acid sequences of the various AAV capsid proteins of AA9 (amino acids 566 to 615 of AAV9 capsid; SEQ ID NO: 30), AAV8 (amino acids 565 to 614 of AAV8 capsid; SEQ ID NO: 31), AAV7 (amino acids 567 to 616 of AAV7; SEQ ID NO: 32), AAV6 (amino acids 550 to 599 of AAV6 capsid; SEQ ID NO: 33), AAV5 (amino acids 556 to 605 of AAV5; SEQ ID NO: 34), AAV4 (amino acids 558 to 607 of AAV4 capsid; SEQ ID NO: 35), AAV3B (amino acids 564 to 613 of AAV3B capsid; SEQ ID NO: 36), AAV2 (amino acids 566 to 615 of AAV2 capsid; SEQ ID NO: 37), and AAV1 (amino acids 566 to 615 of AAV1 capsid; SEQ ID NO: 38), which is focused on the region HVRVIII in which the targeting peptide may be inserted (based on structure analysis).

In certain embodiments, the parental capsid modified to contain the Y-G/A/R/K-Y/H-GNPA-T/R/H-RYFD-V/K (SEQ ID NO: 14: also referred to as Y-X′-X″-GNPA-X″-RYFD-X″, wherein X′ is G, A, R, K, X″ is Y or H, X″ is T, R or H, and X″″ is V or K) core targeting motif, with optional flanking sequences, is selected from parvoviruses which natively target the CNS (e.g., Clade F AAV (e.g., AAVhu68 or AAV9), Clade E (e.g., AAV8), or certain Clade A AAV (e.g., AAV1, AAVrh91)) capsids, or non-parvovirus capsids (e.g., herpes simplex virus, etc.) in order enhance expression and/or otherwise modulate the type of CNS targeted cells. In other embodiments, the capsid is selected from parvoviruses which do not natively target the CNS (e.g., Clade F AAV, e.g., AAVhu68 or AAV9, or certain Clade A AAV, e.g., AAV1, AAVrh91) capsids, or non-parvovirus capsids (e.g., herpes simplex virus (HSV), etc.). See, e.g., WO 2020/223231, published Nov. 5, 2020 (rh91, including table with deamidation pattern), U.S. Provisional Patent Application No. 63/065,616, filed Aug. 14, 2020, U.S. Provisional Patent Application No. 63/109,734, filed Nov. 4, 2020, and International Patent Application No. PCT/US21/45945, filed Aug. 13, 2021, which is now published as WO 2022/036220, all of which are incorporated herein by reference in their entireties. In certain embodiments, AAV capsids having reduced capsid deamidation may be selected. See, e.g., PCT/US19/19804 and PCT/US18/19861, both filed Feb. 27, 2019 and incorporated by reference in their entireties.

In certain embodiments, a recombinant adeno-associated viral particle (rAAV) comprising an AAV capsid, wherein the AAV capsid is not an AAV2 capsid. In certain embodiments, the rAAV comprises an AAV capsid, wherein the AAV capsid is not a mutant AAV2 capsid comprising NDVRAVS (SEQ ID NO: 12) sequence. In certain embodiments, the rAAV comprises an AAV2 capsid wherein the AAV2 capsid protein comprises at least one or more of the exogenous peptides with YGY-GNPA-T/R/H-RYFD-V/K (SEQ ID NO: 14; also referred to as Y-X′-X″-GNPA-X″-RYFD-X″, wherein X′ is G, A, R, K, X″ is Y or H, X″ is T, R or H, and X″ is V or K) core targeting motif. In certain embodiments, the rAAV comprises an AAV2 capsid wherein the AAV2 capsid protein comprises at least one or more of the exogenous peptides with YGY-GNPA-T/R/H-RYFD-V/K (SEQ ID NO: 14) core targeting motif, wherein optionally the core targeting motif is flanked by an N-terminal and/or C-terminal linker sequences. In certain embodiments, the rAAV comprises an AAV9 capsid wherein the AAV9 capsid protein comprises at least one or more of the exogenous peptides with YGY-GNPA-T/R/H-RYFD-V/K (SEQ ID NO: 14) core targeting motif. In certain embodiments, the rAAV comprises an AAV9 capsid wherein the AAV9 capsid protein comprises at least one or more of the exogenous peptides with YGY-GNPA-T/R/H-RYFD-V/K (SEQ ID NO: 14) core targeting motif, wherein optionally the core targeting motif is flanked by an N-terminal and/or C-terminal linker sequences. In certain embodiments, the rAAV comprises an AAVhu68 capsid wherein the AAVhu68 capsid protein comprises at least one or more of the exogenous peptides with YGY-GNPA-T/R/H-RYFD-V/K (SEQ ID NO: 14) core targeting motif. In certain embodiments, the rAAV comprises an AAVhu68 capsid wherein the AAVhu68capsid protein comprises at least one or more of the exogenous peptides with YGY-GNPA-T/R/H-RYFD-V/K (SEQ ID NO: 14) core targeting motif, wherein optionally the core targeting motif is flanked by an N-terminal and/or C-terminal linker sequences.

In certain embodiments, the rAAV comprises an AAV9 capsid wherein the AAV9 capsid protein comprises at least one or more of the exogenous peptides with YGYGNPATRYFDV (“YGY”; SEQ ID NO: 1) core targeting motif. In certain embodiments, the rAAV comprises an AAV9 capsid wherein the AAV9 capsid protein comprises at least one or more of the exogenous peptides with YGYGNPATRYFDV (“YGY”; SEQ ID NO: 1) core targeting motif, wherein optionally the core targeting motif is flanked by an N-terminal and/or C-terminal linker sequences.

In certain embodiments, the rAAV comprises an AAV9 capsid wherein the AAV9 capsid protein comprises at least one or more of the exogenous peptides with YAYGNPATRYFDV (“YGY2A”; SEQ ID NO: 2) core targeting motif. In certain embodiments, the rAAV comprises an AAV9 capsid wherein the AAV9 capsid protein comprises at least one or more of the exogenous peptides with YAYGNPATRYFDV (“YGY2A”; SEQ ID NO: 2) core targeting motif, wherein optionally the core targeting motif is flanked by an N-terminal and/or C-terminal linker sequences.

Thus, provided herein are engineered AAV-9 capsids, including, e.g., an AAV9-YGY capsid, which is expressed from a nucleic acid sequence of SEQ ID NO: 28, or a sequence at least about 70% identical thereto which encodes the amino acid sequence of SEQ ID NO: 29. In certain embodiments, the capsid is AAV9-YGY2A capsid which is expressed from a nucleic acid sequence of SEQ ID NO: 26, or a sequence at least about 70% identical thereto encoding the amino acid sequence of SEQ ID NO: 27.

For example, capsids from Clade F AAV such as AAVhu68 or AAV9 may be selected. Methods of generating vectors having the AAV9 capsid or AAVhu68 capsid, and/or chimeric capsids derived from AAV9 have been described. See, e.g., U.S. Pat. No. 7,906,111, which is incorporated by reference herein. See also, U.S. Provisional Patent Application No. 63/093,275, filed Oct. 18, 2020, which is incorporated herein by reference. Other AAV serotypes which transduce nasal cells or another suitable target (e.g., muscle or lung) may be selected as sources for capsids of AAV viral vectors including, e.g., AAV1, AAV2, AAV3, AAV4, AAV5, AAV6, AAV6.2, AAV7, AAV8, AAV9, rh10, AAVrh64R1, AAVrh64R2, rh8, AAVrh32.33 (See, e.g., US Published Patent Application No. 2007-0036760-A1; US Published Patent Application No. 2009-0197338-A1; and EP 1310571). See also, WO 2003/042397 (AAV7 and other simian AAV), U.S. Pat. Nos. 7,790,449 and 7,282,199 (AAV8), WO 2005/033321 (AAV9), and WO 2006/110689, or yet to be discovered, or a recombinant AAV based thereon, may be used as a source for the AAV capsid. See, e.g., WO 2020/223232 A1 (AAV rh90), WO 2020/223231 A1 International Application No. PCT/US21/45945, filed Aug. 13, 2021 (AAV rh91), and WO 2020/223236 A1 (AAV rh92, AAV rh93, AAV rh91.93), which are incorporated herein by reference in its entirety. These documents also describe other AAV which may be selected for generating AAV and are incorporated by reference. In some embodiments, an AAV capsid (cap) for use in the viral vector can be generated by mutagenesis (i.e., by insertions, deletions, or substitutions) of one of the aforementioned AAV caps or its encoding nucleic acid. In some embodiments, the AAV capsid is chimeric, comprising domains from two or three or four or more of the aforementioned AAV capsid proteins. In some embodiments, the AAV capsid is a mosaic of Vp1, Vp2, and Vp3 monomers from two or three different AAVs or recombinant AAVs. In some embodiments, an rAAV composition comprises more than one of the aforementioned caps.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(12): 6381-6388, which identifies Clades A, B, C, D, E and F, and provides nucleic acid sequences of novel AAV, GenBank Accession Numbers AY530553 to AY530629. See, also, WO 2005/033321.

As used herein, an “AAV9 capsid” is a self-assembled AAV capsid composed of multiple AAV9 vp proteins. The AAV9 vp proteins are typically expressed as alternative splice variants encoded by a nucleic acid sequence which encodes the vp1 amino acid sequence of GenBank accession: AAS99264. These splice variants result in proteins of different length. In certain embodiments, “AAV9 capsid” includes an AAV having an amino acid sequence which is 99% identical to AAS99264 or 99% identical thereto. See, also, WO 2019/168961, published Sep. 6, 2019, including Table G providing the deamidation pattern for AAV9. See, also U.S. Pat. No. 7,906,111 and WO 2005/033321. When specified, “AAV9 variants” may include those described in, e.g., WO2016/049230, U.S. Pat. No. 8,927,514, US 2015/0344911, and U.S. Pat. No. 8,734,809.

A rAAVhu68 is composed of an AAVhu68 capsid and a vector genome. An AAVhu68 capsid is an assembly of a heterogenous population of vp1, a heterogenous population of vp2, and a heterogenous population of vp3 proteins. As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. See, also, PCT/US2018/019992, WO 2018/160582, entitled “Adeno-Associated Virus (AAV) Clade F Vector and Uses Therefor”, and which are incorporated herein by reference in its entirety.

For other recombinant viral vectors, suitable exposed portions of the viral capsid or envelope protein which is responsible for targeting specificity are selected for insertion of the targeting peptide. For example, in an adenovirus, it may be desirable to modify the hexon protein. In a lentivirus, an envelope fusion protein may modified comprise one or more copies of the targeting motif. For vaccinia virus, the major glycoprotein may be modified to comprise one or more copies of the targeting motif. Suitably, these recombinant viral vectors are replication-defective for safety purposes.

### Expression Cassette and Vectors

Vector genomic sequences which are packaged into an AAV capsid and delivered to a host cell are typically composed of, at a minimum, a transgene and its regulatory sequences, and AAV inverted terminal repeats (ITRs). Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue.

The AAV sequences of the vector typically comprise the cis-acting AAV5′ and AAV3″ inverted terminal repeat (ITR) sequences (See, e.g., B. J. Carter, in “Handbook of Parvoviruses”, ed., P. Tijsser, CRC Press, pp. 155 168 (1990)). The ITR sequences are about 145 base pairs (bp) in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences are from AAV2. However, ITRs from other AAV sources may be selected. A shortened version of the 5′ ITR, termed ΔITR, has been described in which the D-sequence and terminal resolution site (trs) are deleted. In certain embodiments, the vector genome includes a shortened AAV2 ITR of 130 base pairs, wherein the external A elements is deleted. Without wishing to be bound by theory, it is believed that the shortened ITR reverts back to the wild-type (WT) length of 145 base pairs during vector DNA amplification using the internal (A ‘) element as a template. In other embodiments, full-length AAV 5’ and 3′ ITRs are used. Where the source of the ITRs is from AAV2 and the AAV capsid is from another AAV source, the resulting vector may be termed pseudotyped. However, other configurations of these elements may be suitable.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

The regulatory control elements typically contain a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be used may be utilized in the vectors described herein.

Examples of constitutive promoters suitable for controlling expression of the therapeutic products include, but are not limited to chickenβ-actin (CB) promoter, CB7 promoter (comprising CMV IE enhancer and CB promoter, optionally linked by a linker sequence), human cytomegalovirus (CMV) promoter, ubiquitin C promoter (UbC), the early and late promoters of simian virus 40 (SV40), U6 promoter, metallothionein promoters, EF1α promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86: 10006-10010 (1989)), the long terminal repeats (LTR) of Moloney Leukemia Virus and other retroviruses, the thymidine kinase promoter of Herpes Simplex Virus and other constitutive promoters known to those of skill in the art. Examples of tissue- or cell-specific promoters suitable for use in the present invention include, but are not limited to, endothelin-I (ET-I) and Flt-I, which are specific for endothelial cells, FoxJ1 (that targets ciliated cells). Other examples of tissue specific promoters suitable for use in the present invention include, but are not limited to, liver-specific promoters. Examples of liver-specific promoters may include, e.g., thyroid hormone-binding globulin (TBG), albumin, Miyatake et al., (1997) J. Virol., 71:5124 32; hepatitis B virus core promoter, Sandig et al., (1996) Gene Ther., 3:1002 9; or human alpha 1-antitrypsin, phosphoenolpyruvate carboxykinase (PECK), or alpha fetoprotein (AFP), Arbuthnot et al., (1996) Hum. Gene Ther., 7:1503 14). In certain embodiments, the promoter is a tissue-specific (e.g., neuron-specific) promoter. In certain embodiments, a suitable promoter may include without limitation, an elongation factor 1 alpha (EF1 alpha) promoter (see, e.g., Kim D W et al, Use of the human elongation factor 1 alpha promoter as a versatile and efficient expression system. Gene. 1990 Jul. 16:91(2):217-23), a Synapsin 1 promoter (see, e.g., Kügler S et al, Human synapsin I gene promoter confers highly neuron-specific long-term transgene expression from an adenoviral vector in the adult rat brain depending on the transduced area. Gene Ther. 2003 February;10(4):337-47), a shorted synapsin promoter, a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February;145(2):613-9. Epub 2003 Oct. 16), or a CB6 promoter (see, e.g., Large-Scale Production of Adeno-Associated Viral Vector Serotype-9 Carrying the Human Survival Motor Neuron Gene, Mol. Biotechnol. 2016 January;58(1):30-6. doi: 10.1007/s12033-015-9899-5). Preferably, such promoters are of human origin.

Inducible promoters suitable for controlling expression of the therapeutic product include promoters responsive to exogenous agents (e.g., pharmacological agents) or to physiological cues. These response elements include, but are not limited to a hypoxia response element (HRE) that binds HIF-1α and β, a metal-ion response element such as described by Mayo et al. (1982, Cell 29:99-108): Brinster et al. (1982, Nature 296:39-42) and Searle et al. (1985, Mol. Cell. Biol. 5:1480-1489); or a heat shock response element such as described by Nouer et al. (in: Heat Shock Response, ed. Nouer, L., CRC, Boca Raton, Fla., pp167-220, 1991).

In one embodiment, expression of the gene product is controlled by a regulatable promoter that provides tight control over the transcription of the sequence encoding the gene product, e.g., a pharmacological agent, or transcription factors activated by a pharmacological agent or in alternative embodiments, physiological cues. Promoter systems that are non-leaky and that can be tightly controlled are preferred. Examples of regulatable promoters which are ligand-dependent transcription factor complexes that may be used in the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch® Mammalian Inducible Expression System (New England Biolabs, Ipswich, MA).

Still other promoter systems may include response elements including but not limited to a tetracycline (tet) response element (such as described by Gossen & Bujard (1992, Proc. Natl. Acad. Sci. USA 89:5547-551); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. Using such promoters, expression of the soluble hACE2 construct can be controlled, for example, by the Tet-on/off system (Gossen et al., 1995, Science 268:1766-9; Gossen et al., 1992, Proc. Natl. Acad. Sci. USA., 89(12):5547-51); the TetR-KRAB system (Urrutia R., 2003, Genome Biol., 4(10):231; Deuschle U et al., 1995, Mol Cell Biol. (4): 1907-14); the mifepristone (RU486) regulatable system (Geneswitch: Wang Y et al., 1994, Proc. Natl. Acad. Sci. USA., 91(17):8180-4: Schillinger et al., 2005, Proc. Natl. Acad. Sci. USA. 102(39):13789-94); and the humanized tamoxifen-dep regulatable system (Roscilli et al., 2002, Mol. Ther. 6(5):653-63).

In another aspect, the gene switch is based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595, U.S. Publication No. 2002/0173474, U.S. Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258, WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety. The Ariad system is designed to be induced by rapamycin and analogs thereof referred to as “rapalogs”. Examples of suitable rapamycins are provided in the documents listed above in connection with the description of the ARGENT™ system. In one embodiment, the molecule is rapamycin [e.g., marketed as Rapamune™ by Pfizer]. In another embodiment, a rapalog known as AP21967 [ARIAD] is used. Examples of these dimerizer molecules that can be used in the present invention include, but are not limited to rapamycin, FK506, FK10¹² (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc Natl Acad Sci USA, 94(20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, e.g., AP23573 [Merck]. In certain embodiments, rapamycin or a suitable analog may be delivered locally to the AAV-transfected cells of the nasopharynx. This local delivery may be by intranasal injection, topically to the cells via bolus, cream, or gel. See, US Patent Application US 2019/0216841 A1, which is incorporated herein by reference.

Other suitable enhancers include those that are appropriate for a desired target tissue indication. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. Other suitable introns include those known in the art, e.g., such as are described in WO 2011/126808. Examples of suitable polyadenylation (polyA) sequences include, e.g., rabbit beta globin (rBG), SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence (see, e.g., MA Zanta-Boussif, et al, Gene Therapy (2009) 16: 605-619).

In certain embodiments, the expression cassette may include one or more expression enhancers such as post-transcriptional regulatory element from hepatitis viruses of woodchuck (WPRE), human (HPRE), ground squirrel (GPRE) or arctic ground squirrel (AGSPRE); or a synthetic post-transcriptional regulatory element. These expression-enhancing elements are particularly advantageous when placed in a 3′ UTR and can significantly increase mRNA stability and/or protein yield. In certain embodiments, the expressions cassettes provided include a regulator sequence that is a woodchuck hepatitis virus posttranscriptional regulatory element (WPRE) or a variant thereof. Suitable WPRE sequences are provided in the vector genomes described herein and are known in the art (e.g., such as those are described in U.S. Pat. Nos. 6,136,597, 6,287,814, and 7,419,829, which are incorporated by reference). In certain embodiments, the WPRE is a variant that has been mutated to eliminate expression of the woodchuck hepatitis B virus X (WHX) protein, including, for example, mutations in the start codon of the WHX gene. In certain embodiments, modified WPRE element is engineered to eliminate expression of the WHX protein, wherein the modified WPRE is a mutated version that contains five-point mutations in the putative promoter region of the WHX gene, along with an additional mutation in the start codon of the WHX gene (ATG mutated to TTG). This mutant WPRE is considered sufficient to eliminate expression of truncated WHX protein based on sensitive flow cytometry analyses of various human cell lines transduced with lentivirus containing a WPRE-GFP fusion construct (Zanta-Boussif et al., 2009). See also, Kingsman S. M., Mitrophanous K., & Olsen J. C. (2005), Potential Oncogene Activity of the Woodchuck Hepatitis Post-Transcriptional Regulatory Element (WPRE).” Gene Ther. 12(1):3-4; and Zanta-Boussif M. A., Charrier S., Brice-Ouzet A., Martin S., Opolon P., Thrasher A. J., Hope T. J., & Galy A. (2009), Validation of a Mutated Pre-Sequence Allowing High and Sustained Transgene Expression While Abrogating Why-X Protein Synthesis: Application to the Gene Therapy of Was, Gene Ther. 16(5):605-19, both of which are incorporated herein by reference in its entirety. In certain embodiments, the WPRE element comprises nucleotides 1093 to 1683 of the GenBank: J02442.1 (591 nucleotides; SEQ ID NO: 40). In certain embodiments, the WPRE element comprises nucleic acid sequence of SEQ ID NO: 39. In other embodiments, enhancers are selected from a non-viral source. In certain embodiments, no WPRE sequence is present.

An AAV viral vector may include multiple transgenes. In certain embodiments, the transgene may be used to correct or ameliorate gene deficiencies, which may include deficiencies in which normal genes are expressed at less than normal levels or deficiencies in which the functional gene product is not expressed. Alternatively, the transgene may provide a product to a cell which is not natively expressed in the cell type or in the host. A preferred type of transgene sequence encodes a therapeutic protein or polypeptide which is expressed in a host cell. The invention further includes using multiple transgenes. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In certain situations, a different transgene may be used to encode each subunit of a protein (e.g., an immunoglobulin domain, an immunoglobulin heavy chain, an immunoglobulin light chain). In one embodiment, a cell produces the multi-subunit protein following infected/transfection with the virus containing each of the different subunits. In another embodiment, different subunits of a protein may be encoded by the same transgene. An IRES is desirable when the size of the DNA encoding each of the subunits is small, e.g., the total size of the DNA encoding the subunits and the IRES is less than five kilobases. As an alternative to an IRES, the DNA may be separated by sequences encoding a 2A peptide, which self-cleaves in a post-translational event. See, e.g., ML Donnelly, et al, (January 1997) J. Gen. Virol., 78(Pt 1): 13-21; S. Furler, S et al, (June 2001) Gene Ther., 8(11):864-873; H. Klump, et al., (May 2001) Gene Ther., 8(10):811-817. This 2A peptide is significantly smaller than IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

In addition to the elements identified above for the expression cassette, the vector also includes conventional control elements which are operably linked to the coding sequence in a manner which permits transcription, translation and/or expression of the encoded product (e.g., UBE3A construct, gene replacement therapy in Angelman mouse models: see, US Provisional Patent Application No. 63/119,860, filed Dec. 1, 2020, which is incorporated herein by reference) in a cell transfected with the plasmid vector or infected with the virus produced by the invention. Examples of other suitable transgenes are provided herein. As used herein, “operably linked” sequences include both expression control sequences that are contiguous with the gene of interest and expression control sequences that act in trans or at a distance to control the gene of interest.

Expression control sequences include appropriate enhancer: transcription factor; transcription terminator; promoter; efficient RNA processing signals such as splicing and polyadenylation (polyA) signals; sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product. In one embodiment, the regulatory sequences are selected such that the total rAAV vector genome is about 2.0 to about 5.5 kilobases in size. In one embodiment, it is desirable that the rAAV vector genome approximate the size of the native AAV genome. Thus, in one embodiment, the regulatory sequences are selected such that the total rAAV vector genome is about 4.7 kb in size. In another embodiment, the total rAAV vector genome is less about 5.2 kb in size. The size of the vector genome may be manipulated based on the size of the regulatory sequences including the promoter, enhancer, intron, poly A, etc. See, Wu et al, Mol Ther, January 2010 18(1):80-6, which is incorporated herein by reference.

Thus, in one embodiment, an intron is included in the vector. Suitable introns include chicken beta-actin intron, the human beta globin IVS2 (Kelly et al, Nucleic Acids Research, 43(9):4721-32 (2015)); the Promega chimeric intron (Almond, B. and Schenborn, E. T. A Comparison of pCI-neo Vector and pcDNA4/HisMax Vector); and the hFIX intron. Various introns suitable herein are known in the art and include, without limitation, those found at bpg.utoledo.edu/˜afedorov/lab/eid.html, which is incorporated herein by reference. See also, Shepelev V., Fedorov A. Advances in the Exon-Intron Database. Briefings in Bioinformatics 2006, 7: 178-185, which is incorporated herein by reference.

In certain embodiments, the mutant rAAV comprises an expression cassette which further comprising at least one miRNA target sequences operably linked to a selected transgene, optionally in its 3′ UTR and/or its 5′ UTR. In certain embodiments, the miRNA is a dorsal root ganglion (drg)-specific miRNA target sequence. In certain embodiments, the nucleic acid sequence further comprises at least one, at least two, at least three or preferably at least four tandem repeats of dorsal root ganglion (drg)-specific miRNA target sequences. In certain embodiments, the nucleic acid sequence further comprises at least one, at least two, at least three, at least four, at least five, at least six, at least seven or at least eight tandem repeats of dorsal root ganglion (drg)-specific miRNA target sequences. See, e.g., PCT/US19/67872, filed Dec. 20, 2019, and now published as WO 2020/132455, which are incorporated herein by reference. See, also, International Patent Application No. PCT/US21/32003, filed May 12, 2021, and now published WO2021/231579A1, which are incorporated herein by reference. See also, U.S. Provisional Patent Application No. 63/279,561, filed Nov. 15, 2021, which is incorporated herein by reference in its entirety.

Several different viral genomes were generated in the studies described herein. However, it will be understood by the skilled artisan that other genomic configurations, including other regulatory sequences may be substituted for the promoter, enhancer and other coding sequences may be selected.

rAAV Vector Production

For use in producing an AAV viral vector (e.g., a recombinant (r) AAV), the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

In certain embodiments, the inclusion of the at least one copy of the Y-G/A/R/K-Y/H-GNPA-T/R/H-RYFD-V/K core targeting motif (also referred as Y-X′-X″-GNPA-X″-RYFD-X′″ motif, wherein X′ is G, A, R, K, X″ is Y or H, X′″ is T, R or H, and X″″ is V or K; SEQ ID NO: 14) into an AAV capsid provides advantages in production as compared to the method without inclusion of at least one copy of motif in AAV capsid, and wherein the production cells are 293 cells. In certain embodiments, nucleic acid sequence encoding an AAV capsid used for production of a recombinant AAV comprising rep sequences and cap sequences comprises SEQ ID NO: 18 or a sequence at least 90%, 95%, 98%, 99%. 99.9%, 100% (or any values therebetween) identical to SEQ ID NO: 18 (i.e., comprising AAV2 rep and AAV9-YGY-modified cap). In certain embodiments, nucleic acid sequence encoding an AAV capsid used for production of a recombinant AAV comprising rep sequences and cap sequences comprises SEQ ID NO: 21 or a sequence at least 90%, 95%, 98%, 99%. 99.9%, 100% (or any values therebetween) identical to SEQ ID NO: 21 (i.e., comprising AAV2 rep and AAV9-YGY2A-modified cap).

Methods of preparing AAV-based vectors (e.g., having an AAV9 or another AAV capsid) are known. See, e.g., US Published Patent Application No. 2007/0036760 (Feb. 15, 2007), which is incorporated by reference herein. The invention is not limited to the use of AAV9 or other clade F AAV amino acid sequences, but encompasses peptides and/or proteins containing the terminal β-galactose binding generated by other methods known in the art, including, e.g., by chemical synthesis, by other synthetic techniques, or by other methods. The sequences of any of the AAV capsids provided herein can be readily generated using a variety of techniques. Suitable production techniques are well known to those of skill in the art. See, e.g., Sambrook et al, Molecular Cloning: A Laboratory Manual, Cold Spring Harbor Press (Cold Spring Harbor, NY). Alternatively, peptides can also be synthesized by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These methods may involve, e.g., culturing a host cell which contains a nucleic acid sequence encoding an AAV capsid; a functional rep gene: a minigene composed of, at a minimum, AAV inverted terminal repeats (ITRs) and a transgene; and sufficient helper functions to permit packaging of the minigene into the AAV capsid protein. These and other suitable production methods are within the knowledge of those of skill in the art and are not a limitation of the present invention.

The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

These rAAVs are particularly well suited to gene delivery for therapeutic purposes and for preventing infection. Further, the compositions of the invention may also be used for production of a desired gene product in vitro. For in vitro production, a desired product (e.g., a protein) may be obtained from a desired culture following transfection of host cells with a rAAV containing the molecule encoding the desired product and culturing the cell culture under conditions which permit expression. The expressed product may then be purified and isolated, as desired. Suitable techniques for transfection, cell culturing, purification, and isolation are known to those of skill in the art. Methods for generating and isolating AAVs suitable for use as vectors are known in the art. See generally, e.g., Grieger & Samulski, 2005, “Adeno-associated virus as a gene therapy vector: Vector development, production and clinical applications,” Adv. Biochem. Engin/Biotechnol. 99: 119-145: Buning et al., 2008, “Recent developments in adeno-associated virus vector technology,” J. Gene Med. 10:717-733; and the references cited below, each of which is incorporated herein by reference in its entirety. For packaging a transgene into virions, the ITRs are the only AAV components required in cis in the same construct as the nucleic acid molecule containing the expression cassettes. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. Alternatively, the expression cassettes may be used to generate a viral vector other than AAV, or for production of mixtures of antibodies in vitro. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant AAV described herein may be generated using techniques which are known. See, e.g., WO 2003/042397: WO 2005/033321, WO 2006/110689: U.S. Pat. No. 7,588,772 B2. Such a method involves culturing a host cell which contains a nucleic acid sequence encoding an AAV capsid; a functional rep gene: an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, cells are manufactured in a suitable cell culture (e.g., HEK 293 cells). Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest for packaging into the capsid, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, which is incorporated herein by reference in its entirety. Methods of making and using these and other AAV production systems are also described in the following U.S. patents, the contents of each of which is incorporated herein by reference in its entirety: 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in International Patent Application No. PCT/US2016/065970, filed Dec. 9, 2016, entitled “Scalable Purification Method for AAV9”, which is incorporated by reference. Purification methods for AAV8, International Patent Application No. PCT/US2016/065976, filed Dec. 9, 2016, and rh10, International Patent Application No. PCT/US16/066013, filed Dec. 9, 2016, entitled “Scalable Purification Method for AAVrh10”, also filed Dec. 11, 2015, and for AAV1, International Patent Application No. PCT/US2016/065974, filed Dec. 9, 2016 for “Scalable Purification Method for AAV1”, filed Dec. 11, 2015, are all incorporated by reference herein.

To calculate empty and full particle content, vp3 band volumes for a selected sample (e.g., in examples herein an iodixanol gradient-purified preparation where number of GC=number of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6:1322-1330; and Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

Additionally, another example of measuring empty to full particle ratio is also known in the art. Sedimentation velocity, as measured in an analytical ultracentrifuge (AUC) can detect aggregates, other minor components as well as providing good quantitation of relative amounts of different particle species based upon their different sedimentation coefficients. This is an absolute method based on fundamental units of length and time, requiring no standard molecules as references. Vector samples are loaded into cells with 2-channel charcoal-epon centerpieces with 12 mm optical path length. The supplied dilution buffer is loaded into the reference channel of each cell. The loaded cells are then placed into an AN-60Ti analytical rotor and loaded into a Beckman-Coulter ProteomeLab XL-I analytical ultracentrifuge equipped with both absorbance and RI detectors. After full temperature equilibration at 20° C. the rotor is brought to the final run speed of 12,000 rpm. A280 scans are recorded approximately every 3 minutes for ˜5.5 hours (110 total scans for each sample). The raw data is analyzed using the c(s) method and implemented in the analysis program SEDFIT. The resultant size distributions are graphed and the peaks integrated. The percentage values associated with each peak represent the peak area fraction of the total area under all peaks and are based upon the raw data generated at 280 nm; many labs use these values to calculate empty; full particle ratios. However, because empty and full particles have different extinction coefficients at this wavelength, the raw data can be adjusted accordingly. The ratio of the empty particle and full monomer peak values both before and after extinction coefficient-adjustment is used to determine the empty-full particle ratio.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay. Quantification also can be done using ViroCyt or flow cytometry.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April;25(2): 115-25. doi: 10.1089/hgtb.2013.131. Epub 2014 February 14.

### Therapeutic Proteins and Delivery Systems

Fusion partners, conjugate partners and recombinant vectors containing the targeting motif provided herein, Y-G/A/R/K-Y/H-GNPA-T/R/H-RYFD-V/K (also referred as Y-X′-X″-GNPA-X″-RYFD-X″, wherein X′ is G, A, R, K, X″ is Y or H, X″ is T, R or H, and X″ is V or K; SEQ ID NO″ 14) core targeting motif, are useful with a variety of different therapeutic proteins, polypeptides, nanoparticles, and delivery systems. Examples of proteins and compounds useful in compositions provided herein and targeted delivery include the following. It will be understood that the viral vectors, nanoparticles and other delivery systems contain sequences encoding the selected proteins (or conjugates) for expression in vivo.

In some embodiments, the rAAV having a modified capsid with at least one core one or more of core Y-G/A/R/K-Y/H-GNPA-T/R/H-RYFD-V/K (also referred as Y-X-X″-GNPA-X″-RYFD-X″, wherein X′ is G, A, R, K, X″ is Y or H, X′″ is T, R or H, and X″ is V or K; SEQ ID NO″ 14) peptide, comprises vector genome comprising the desired transgene and promoter for use in the target cells as detailed above is optionally assessed for contamination by conventional methods and then formulated into a pharmaceutical composition intended for administration to a subject in need thereof. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid.

Examples of proteins and compounds useful in compositions provided herein and targeted delivery include the following. It will be understood that the rAAV comprise sequences encoding the selected proteins for expression in vivo.

In certain embodiments, proteins, polypeptides, nanoparticles, and/or delivery systems including viral vectors and nanoparticles, comprising the targeting motif provided herein, are useful in treatment of one or more of cognitive disorders and/or neurodegenerative disorders. Such disorders may include, without limitation, transmissible spongiform encephalopathies (e.g., Creutzfeld-Jacob disease), Parkinson's disease, amyotropic lateral sclerosis (ALS), multiple sclerosis, Alzheimer's Disease, Huntington disease, Canavan's disease, traumatic brain injury, spinal cord injury (ATI335, anti-nogol by Novartis), migraine (ALD403 by Alder Biopharmaceuticals: LY2951742 by Eli: RN307 by Labrys Biologics), bovine spongiform encephalopathy, Gerstmann-Sträussler-Scheinker syndrome, fatal familial insomnia, kuruysosomal storage diseases, stroke, and infectious disease affecting the central nervous system.

In certain embodiments, proteins, polypeptides, nanoparticles, and/or delivery systems including viral vectors and nanoparticles, comprising the targeting motif provided herein, are useful in delivery of antibodies against various infections of the central nervous. Such infectious diseases may include fungal diseases such as cryptoccocal meningitis, brain abscess, spinal epidural infection caused by, e.g., Cryptococcus neoformans, Coccidioides immitis, order Mucorales, Aspergillus spp, and Candida spp: protozoal, such as toxoplasmosis, malaria, and primary amoebic meningoencepthalitis, caused by agents such as, e.g., Toxoplasma gondii, Taenia solium, Plasmodium falciparus, Spirometra mansonoides (sparaganoisis), Echinococcus spp (causing neuro hydatosis), and cerebral amoebiasis: bacterial, such as, e.g., tuberculosis, leprosy, neurosyphilis, bacterial meningitis, lyme disease (Borrelia burgdorferi), Rocky Mountain spotted fever (Rickettsia rickettsia), CNS nocardiosis (Nocardia spp), CNS tuberculosis (Mycobacterium tuberculosis), CNS listeriosis (Listeria monocytogenes), brain abscess, and neuroborreliosis: viral infections, such as, e.g., viral meningitis, Eastern equine encephalitis (EEE), St Louis encepthalitis, West Nile virus and/or encephalitis, rabies, California encephalitis virus, La Crosse encepthalitis, measles encephalitis, poliomyelitis, which may be caused by, e.g., herpes family viruses (HSV), HSV-1, HSV-2 (neonatal herpes simplex encephalitis), varicella zoster virus (VZV), Bickerstaff encephalitis, Epstein-Barr virus (EBV), cytomegalovirus (CMV, such as TCN-202 is in development by Theraclone Sciences), human herpesvirus 6 (HHV-6), B virus (herpesvirus simiae), Flavivirus encephalitis, Japanese encephalitis, Murray valley fever, JC virus (progressive multifocal leukoencephalopathy), Nipah Virus (NiV), measles (subacute sclerosing panencephalitis); and other infections, such as, e.g., subactuate sclerosing panencephalitis, progressive multifocal leukoencephalopathy; human immunodeficiency virus (acquired immunodeficiency syndrome (AIDS)); Streptococcus pyogenes and other β-hemolytic Streptococcus (e.g., Pediatric Autoimmune Neuropsychiatric Disorders Associated with Streptococcal Infection, PANDAS) and/or Syndenham's chorea, and Guillain-Barre syndrome, and prions.

In certain embodiments, the protein is MCT8 protein (SLC16A2 gene) and other compounds for treating of Allan-Herndon-Dudley disease and the symptoms thereof.

In certain embodiments, the protein is selected from a disease associated with a transport defect such as, e.g., cystic fibrosis (a cystic fibrosis transmembrane regulator), alpha-1-antitrypsin (hereditary emphysemsa), FE (hereditary hemaochromatosis), tyrosinase (oculocutaneous albinism), Protein C (protein C deficiency), Complement C inhibitor (type I hereditary angioedema), alpha-D-galactosidase (Fabry disease), beta hexosaminidase (Tay-Sachs), sucrase-isomaltase (congenital sucrase-isomaltase deficiency), UDP-glucoronosyl-transferase (Crigler-Najjar type II), insulin receptor (diabetes mellitus), growth hormone receptor (laron syndrome), among others. Examples of other genes and proteins those associated with, e.g. spinal muscular atrophy (SMA, SMN1), Huntingdon's Disease, Rett Syndrome (e.g., methyl-CpG-binding protein 2 (MeCP2); UniProtKB-P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), CDKL5 deficiency, Angelman syndrome, N-glycanase 1 deficiency, Alzheimer's disease, Fragile X syndrome, Neimann Pick disease (including types A and B (ASMD or Acid Sphingomyelinase Deficiency), and type c (NPC), mucopolysaccharidoses (MPS), Wolman disease, among others. See, e.g., orpha.net/consor/cgi-bin/Disease_Search_List.php; rarediseases.info.nih.gov/diseases. Further illustrative genes which may be delivered via the rAAV include, without limitation, glucose-6-phosphatase, associated with glycogen storage disease or deficiency type 1A (GSD1), phosphoenolpyruvate-carboxykinase (PEPCK), associated with PEPCK deficiency: cyclin-dependent kinase-like 5 (CDKL5), also known as serine/threonine kinase 9 (STK9) associated with seizures and severe neurodevelopmental impairment: galactose-1 phosphate uridyl transferase, associated with galactosemia; phenylalanine hydroxylase (PAH), associated with phenylketonuria (PKU): gene products associated with Primary Hyperoxaluria Type 1 including Hydroxyacid Oxidase 1 (GO/HAO1) and AGXT, branched chain alpha-ketoacid dehydrogenase, including BCKDH. BCKDH-E2, BAKDH-E1α, and BAKDH-Elb, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASSI), associated with citrullinemia; lecithin-cholesterol acyltransferase (LCAT) deficiency; amethylmalonic acidemia (MMA); NPCI associated with Niemann-Pick disease, type C1); propionic academia (PA); TTR associated with Transthyretin (TTR)-related Hereditary Amyloidosis, low density lipoprotein receptor (LDLR) protein, associated with familial hypercholesterolemia (FH). LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPDI) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease.

In certain embodiments, the protein is encoded by a transgene sequence including hormones and growth and differentiation factors including, without limitation, insulin, glucagon, glucagon-like peptide 1 (GLP-1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), any one of the transforming growth factor β superfamily, including TGF β, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), lysosomal acid lipase (LIPA or LAL), neurturin, agrin, any one of the family of semaphorins/collapsins, netrin-1 and netrin-2, hepatocyte growth factor (HGF), ephrins, noggin, sonic hedgehog and tyrosine hydroxylase. Other useful transgene encode lysosomal enzymes that cause mucopolysaccharidoses (MPS), including α-L-iduronidase (MPSI), iduronate sulfatase (MPSII), heparan N-sulfatase (sulfaminidase) (MPS IIIA, Sanfilippo A), α-N-acetyl-glucosaminidase (MPS IIIB, Sanfilippo B), acetyl-CoA;α-glucosaminide acetyltransferase (MPS IIIC, Sanfilippo C), N-acetylglucosamine 6-sulfatase (MPS IIID, Sanfilippo D), galactose 6-sulfatase (MPS IVA, Morquio A), β-Galactosidase (MPS IVB, Morquio B), N-acetyl-galactosamine 4-sulfatase (MPS VI, Maroteaux-Lamy), β-Glucuronidase (MPS VII, Sly), and hyaluronidase (MPS IX).

In certain embodiments, the protein is encoded by a transgene sequence including a reporter sequence, which upon expression produces a detectable signal. Such reporter sequences include, without limitation, DNA sequences encoding β-lactamase, β-galactosidase (LacZ), alkaline phosphatase, thymidine kinase, green fluorescent protein (GFP), enhanced GFP (EGFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, the protein is selected from a disease associated the indicated neurological diseases, disorders, syndrome and/or conditions include, but not limited to, spinal muscular atrophy (SMA) (associated with survival motor neuron protein (SMN2) gene), SMN1, amyotrophic lateral sclerosis (ALS) (superoxide dismutase type 1 (SOD1), FUS RNA binding protein (FUS), microRNA-155, chromosome 9 open reading frame 72 (C9orf72), or ataxin-2 (ATXN2) genes), Huntington disease (associated with huntingtin (HTT) gene), hATTR polyneuropathy (associated with transthyretin (TTR) gene), Alzheimer's disease (associated with MAP-tau (MAPT) gene), Multiple System Atrophy (associated with alpha-synuclein (SNCA)), Parkinson's disease (associated with alpha-synuclein (SNCA), leucine rich repeat kinase 2 (LRRK2) genes), centronuclear myopathy (associated with dynamin 2 (DNM2) gene), Angelman syndrome (associated with ubiquitin protein ligase E3A (UBE3A) gene), epilepsy (associated with glycogen synthase 1 (GYS1) gene), Dravet Syndrome (associated with sodium voltage-gated channel alpha subunit 1 (SNCIA) gene), Leukodystrophy (associated with glial fibrillary acidic protein (GFAP) gene), prion disease (associated with prion protein (PRNP) gene), and Hereditary cerebral hemorrhage with amyloidosis-Dutch type (HCHWA-D) (associated with amyloid beta precursor protein (APP) gene).

An rAAV having a mutant rAAV capsid as provided herein has a vector genome which comprises nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi or miRNA), antisense oligonucleotides etc. These may be in additional to or in alternative to a protein to be delivered.

### Compositions and Uses

Provided herein are compositions containing at least one rAAV stock (e.g., an rAAV9 mutant stock or rAAVhu68 mutant stock, wherein mutant comprises a core targeting motif as described herein) and an optional carrier, excipient and/or preservative. An rAAV stock refers to a plurality of rAAV vectors which are the same, e.g., such as in the amounts described below in the discussion of concentrations and dosage units.

Provided herein, also, are compositions containing at least one therapeutic protein, polypeptide, nanoparticles and/or delivery system comprising the targeting motif as provided herein, and an optional carrier, excipient and/or preservative.

Provided herein, also, are methods of use of compositions as described herein. In certain embodiments, a method for targeted therapy to brain cells comprising administering to a patient in need thereof a stock of the rAAV as described herein, wherein a therapeutic is targeted for delivery to cells in brain and/or spinal cord, and is de-targeted for cells in liver, heart and/or lung.

Additionally, provided herein is a method of delivering of a transgene to one or more target cells of the central nervous system (CNS) of a subject comprising administering to the subject a recombinant adeno-associated virus (AAV) vector comprising modified capsid with at least one core one or more of core Y-G/A/R/K-Y/H-GNPA-T/R/H-RYFD-V/K (also referred as Y-X′-X″-GNPA-X″-RYFD-X″, wherein X′ is G, A, R, K, X″ is Y or H, X″ is T, R or H, and X″ is V or K; SEQ ID NO″ 14) peptide, and a vector genome comprising the transgene operably linked to regulatory sequences that direct expression of the transgene in the target cells of the CNS. In certain embodiments, the target cells of the CNS are parenchymal cells, cells of the choroid plexus, ependymal cells, astrocytes, and/or and neurons, optionally neurons of the cortex, hippocampus, and/or striatum. In certain embodiments, the transgene encodes a secreted gene product. In certain embodiments, the AAV vector is delivered intrathecally, optionally via intra-cisterna magna (ICM) injection. In certain embodiments, the AAV vector is delivered via intraparenchymal administration. In certain embodiments, the AAV vector is delivered via Ommaya Reservoir delivery system.

Provided herein are also uses of an rAAV having a modified capsid with at least one core one or more of core Y-G/A/R/K-Y/H-GNPA-T/R/H-RYFD-V/K (also referred as Y-X′-X″-GNPA-X″-RYFD-X″, wherein X′ is G, A, R, K, X″ is Y or H, X″ is T, R or H, and X″ is V or K; SEQ ID NO″ 14) peptide, to target cells of the brain, such as astrocytes, at higher levels of transduction than achieved using an AAV9 vector. In certain embodiments, higher transduction levels are achieved in caudal sections of the brain, including frontal and temporal cortices. In certain embodiments, an rAAV having a modified capsid with at least one core one or more of core Y-G/A/R/K-Y/H-GNPA-T/R/H-RYFD-V/K peptide (also referred as Y-X′-X″-GNPA-X″-RYFD-X″, wherein X′ is G, A, R, K, X″ is Y or H, X″ is T, R or H, and X″ is V or K; SEQ ID NO″ 14) achieves higher levels of transduction, for example relative to AAV9, of neurons in the cortex, hippocampus, and/or striatum.

In certain embodiments, a composition may contain at least a second, different rAAV stock. This second vector stock may vary from the first by having a different AAV capsid and/or a different vector genome. In certain embodiments, a composition as described herein may contain a different vector expressing an expression cassette as described herein, or another active component (e.g., an antibody construct, another biologic, and/or a small molecule drug).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Suitably, the final formulation is adjusted to a physiologically acceptable pH, e.g., the pH may be in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of 6.8 to about 7.2 may be desired. However, other pHs within the broadest ranges and these subranges may be selected for other routes of delivery. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among nonionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits; the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one embodiment, the compositions described herein are used in preparing medicaments for treating central nervous system disorders and diseases. Optionally, the compositions described herein are administered in the absence of an additional extrinsic pharmacological or chemical agent, or other physical disruption of the blood brain barrier.

In one embodiment, the formulation buffer is phosphate-buffered saline (PBS) with total salt concentration of 200 mM, 0.001% (w/v) pluronic F68 (Final Formulation Buffer, FFB).

In certain embodiments, the composition comprises a viral vector (i.e., rAAV vector). The vectors are administered in sufficient amounts to transfect the cells and to provide sufficient levels of gene transfer and expression to provide a therapeutic benefit without undue adverse effects, or with medically acceptable physiological effects, which can be determined by those skilled in the medical arts. In certain embodiments, the vectors are formulated for delivery via intranasal delivery devices. In certain embodiments, vectors are formulated for aerosol delivery devices, e.g., via a nebulizer or through other suitable devices. In certain embodiment, vectors are formulated for intrathecal delivery. In some embodiments, intrathecal delivery encompasses an injection into the spinal canal, e.g., the subarachnoid space. In some embodiments, other delivery route may be selected, e.g., intracranial, intranasal, intracisternal, intracerebrospinal fluid delivery, among other suitable direct or systemic routes, i.e., Ommaya reservoir. In certain embodiments, vectors are formulated for intravenous delivery. Other conventional and pharmaceutically acceptable routes of administration include, but are not limited to, direct delivery to a desired organ (e.g., lung), oral inhalation, intratracheal, intraarterial, intraocular, intravenous, intramuscular, subcutaneous, intradermal, and other parenteral routes of administration. In one embodiment, the vector is administered intranasally using intranasal mucosal atomization device (LMAR, MAD Nasal™-MAD110). In another embodiment the vector is administered intrapulmonary in nebulized form using Vibrating Mesh Nebulizer (Acrogen R, Solo) or MADgic™ Laryngeal Mucosal Atomizer. Routes of administration may be combined, if desired. Routes of administration and utilization of which for delivering rAAV vectors are also described in the following published US patent applications, the contents of each of which is incorporated herein by reference in its entirety; US 2018/0155412A1, US 2018/0243416A1, US 2014/0031418 A1, and US 2019/0216841A1.

Dosages of the viral vector will depend primarily on factors such as the condition being treated, the age, weight and health of the patient, and may thus vary among patients. For example, a therapeutically effective human dosage of the viral vector is generally in the range of from about 25 to about 1000 microliters to about 5 mL of aqueous suspending liquid containing doses of from about 10⁹ to 4×10¹⁴ GC of AAV vector. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in viral vectors, preferably AAV vectors containing the minigene. Optionally, dosage regimens similar to those described for therapeutic purposes may be utilized for immunization using the compositions of the invention.

In certain embodiments, the compositions can be formulated in dosage units to contain an amount of rAAV that is in the range of about 1×10⁹ GC per gram of brain mass to about 1×10¹³ genome copies (GC) per gram (g) of brain mass, including all integers or fractional amounts within the range and the endpoints. In another embodiment, the dosage is 1×10¹⁰ GC per gram of brain mass to about 1×10¹³ GC per gram of brain mass. In specific embodiments, the dose of the vector administered to a patient is at least about 1.0×10⁹ GC/g, about 1.5×10⁹ GC/g, about 2.0×10⁹ GC/g, about 2.5×10⁹ GC/g, about 3.0×10⁹ GC/g, about 3.5×10⁹ GC/g, about 4.0×10⁹ GC/g, about 4.5×10⁹ GC/g, about 5.0×10⁹ GC/g, about 5.5×10⁹ GC/g, about 6.0×10⁹ GC/g, about 6.5×10⁹ GC/g, about 7.0×10⁹ GC/g, about 7.5×10⁹ GC/g, about 8.0×10⁹ GC/g, about 8.5×10⁹ GC/g, about 9.0×10⁹ GC/g, about 9.5×10⁹ GC/g, about 1.0×10¹⁰ GC/g, about 1.5×10¹⁰ GC/g, about 2.0×10¹⁰ GC/g, about 2.5×10¹⁰ GC/g, about 3.0×10¹⁰ GC/g, about 3.5×10¹⁰ GC/g, about 4.0×10¹⁰ GC/g, about 4.5×10¹⁰ GC/g, about 5.0×10¹⁰ GC/g, about 5.5×10¹⁰ GC/g, about 6.0×10¹⁰ GC/g, about 6.5×10¹⁰ GC/g, about 7.0×10¹⁰ GC/g, about 7.5×10¹⁰ GC/g, about 8.0×10¹⁰ GC/g, about 8.5×10¹⁰ GC/g, about 9.0×10¹⁰ GC/g, about 9.5×10¹⁰ GC/g, about 1.0×10¹¹ GC/g, about 1.5×10¹¹ GC/g, about 2.0×10¹¹ GC/g, about 2.5×10¹¹ GC/g, about 3.0×10¹¹ GC/g, about 3.5×10¹¹ GC/g, about 4.0×10¹¹ GC/g, about 4.5×10¹¹ GC/g, about 5.0×10¹¹ GC/g, about 5.5×10¹¹ GC/g, about 6.0×10¹¹ GC/g, about 6.5×10¹¹ GC/g, about 7.0×10¹¹ GC/g, about 7.5×10¹¹ GC/g, about 8.0×10¹¹ GC/g, about 8.5×10¹¹ GC/g, about 9.0×10¹¹ GC/g, about 9.5×10¹¹ GC/g, about 1.0×10¹² GC/g, about 1.5×10¹² GC/g, about 2.0×10¹² GC/g, about 2.5×10¹² GC/g, about 3.0×10¹² GC/g, about 3.5×10¹² GC/g, about 4.0×10¹² GC/g, about 4.5×10¹² GC/g, about 5.0×10¹² GC/g, about 5.5×10¹² GC/g, about 6.0×10¹² GC/g, about 6.5×10¹² GC/g, about 7.0×10¹² GC/g, about 7.5×10¹² GC/g, about 8.0×10¹² GC/g, about 8.5×10¹² GC/g, about 9.0×10¹² GC/g, about 9.5×10¹² GC/g, about 1.0×10¹³ GC/g, about 1.5×10¹³ GC/g, about 2.0×10¹³ GC/g, about 2.5×10¹³ GC/g, about 3.0×10¹³ GC/g, about 3.5×10¹³ GC/g, about 4.0×10¹³ GC/g, about 4.5×10¹³ GC/g, about 5.0×10¹³ GC/g, about 5.5×10¹³ GC/g, about 6.0×10¹³ GC/g, about 6.5×10¹³ GC/g, about 7.0×10¹³ GC/g, about 7.5×10¹³ GC/g, about 8.0×10¹³ GC/g, about 8.5×10¹³ GC/g, about 9.0×10¹³ GC/g, about 9.5×10¹³ GC/g, or about 1.0×10¹⁴ GC/g brain mass.

The replication-defective virus compositions can be formulated in dosage units to contain an amount of replication-defective virus that is in the range of about 10⁹ GC to about 10¹⁶ GC (to treat an average subject of 70 kg in body weight) including all integers or fractional amounts within the range, and preferably 10¹² GC to 10¹⁴ GC for a human patient. In one embodiment, the compositions are formulated to contain at least 10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 10¹⁰ to about 10¹² GC per dose including all integers or fractional amounts within the range. In one embodiment, for human application the dose can range from 10⁹ to about 7×10¹³ GC per dose including all integers or fractional amounts within the range. In one embodiment, for human application the dose ranges from 6.25×10¹² GC to 5.00×10¹³ GC. In a further embodiment, the dose is about 6.25×10¹² GC, about 1.25×10¹³ GC, about 2.50×10¹³ GC, or about 5.00×10¹³ GC. In certain embodiment, the dose is divided into one half thereof equally and administered to each nostril. In certain embodiments, for human application the dose ranges from 6.25×10¹² GC to 5.00×10¹³ GC administered as two aliquots of 0.2 ml per nostril for a total volume delivered in each subject of 0.8 ml.

These above doses may be administered in a variety of volumes of carrier, excipient or buffer formulation, ranging from about 25 to about 1000 microliters, or higher volumes, including all numbers within the range, depending on the size of the area to be treated, the viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In certain embodiments, the dose may be in the range of about 1×10⁹ GC/g brain mass to about 1×10¹² GC/g brain mass. In certain embodiments, the dose may be in the range of about 3×10¹⁰ GC/g brain mass to about 3×10¹¹ GC/g brain mass. In certain embodiments, the dose may be in the range of about 5×10¹⁰ GC/g brain mass to about 1.85×10¹¹ GC/g brain mass.

In one embodiment, the viral constructs may be delivered in doses of from at least about least 1×10⁹ GCs to about 1×10¹⁵, or about 1×10¹¹ to 5×10¹³ GC. Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, Ommaya reservoir device, or for delivery by another device or route. In one example, the composition is formulated for intrathecal delivery.

The composition, the suspension or the pharmaceutical compositions described herein are designed for delivery to subjects in need thereof by any suitable route or a combination of different routes. In one embodiment, the pharmaceutical composition comprises a formulation buffer suitable for delivery via intracerebroventricular (ICV), intrathecal (IT), intracisternal, administration by direct injection into the substantia nigra and/or ventral tegmental area, or intravenous (IV) routes of administration. In certain embodiments, the rAAV or the pharmaceutical composition comprises a formulation buffer suitable for intravenous, intraparenchymal (dentate nucleus) and/or intrathecal administration to a patient in the need thereof.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, intracerebroventricular (icv) suboccipital/intracisternal, and/or C1-2 puncture. For example, material may be introduced for diffusion throughout the subarachnoid space by means of lumbar puncture. In another example, injection may be into the cisterna magna (intracisternal magna: ICM). Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther Methods Clin Dev. 2014; 1: 14051. Published online 2014 Dec. 10. doi: 10.1038/mtm.2014.51.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

As used herein, the term “intraparenchymal (dentate nucleus)” or IDN refers to a route of administration of a composition directly into dentate nuclei. IDN allows for targeting of dentate nuclei and/or cerebellum. In certain embodiments, the IDN administration is performed using ClearPoint® Neuro Navigation System (MRI Interventions, Inc., Memphis, TN) and ventricular cannula, which allows for MRI-guided visualization and administration. Alternatively, other devices and methods may be selected.

In one embodiment, a frozen composition is provided which contains an rAAV in a buffer solution as described herein, in frozen form. Optionally, one or more surfactants (e.g., Pluronic F68), stabilizers or preservatives is present in this composition. Suitably, for use, a composition is thawed and titrated to the desired dose with a suitable diluent, e.g., sterile saline or a buffered saline.

In one embodiment, a composition comprising one or more exogenous brain cell-targeting peptide(s) from the core: Y-G/A/R/K-Y/H-GNPA-T/R/H-RYFD-V/K (also referred as Y-X-X″-GNPA-X″-RYFD-X″ motif, wherein X′ is G, A, R, K, X″ is Y or H, X″ is T, R or H, and X″ is V or K; SEQ ID NO: 14) and optional flanking linker sequences are provided, together with one or more of a physiologically compatible carrier, excipient, and/or aqueous suspension base. Further provided are compositions comprising nucleic acid sequences encoding same. In certain embodiments, the targeting peptide core amino acid sequence is of SEQ ID NO: 1 and is encoded by a nucleic acid sequence of SEQ ID NO: 25, or a sequence at least about 70% identical thereto. In certain embodiments, the targeting peptide core amino acid sequence is of SEQ ID NO: 2 and is encoded by a nucleic acid sequence of SEQ ID NO: 24, or a sequence at least about 70% identical thereto.

In another embodiment, a fusion polypeptide or protein is provided comprising one or more exogenous brain cell-targeting peptide core(s) from the targeting motif: Y-G/A/R/K-Y/H-GNPA-T/R/H-RYFD-V/K (SEQ ID NO: 14) are provided and fusion partner which comprises at least one polypeptide or protein. Further provided are nucleic acid sequences encoding same.

In certain embodiments, a composition comprising a fusion polypeptide or protein, or a nucleic acid sequence encoding the fusion polypeptide or protein, or a nanoparticle containing same are provided. The composition may further comprise one or more of a physiologically compatible carrier, excipient, and/or aqueous suspension base.

In certain embodiments, a nucleic acid sequence encoding the fusion polypeptide protein is encapsulated in a lipid nanoparticle (LNP). As used herein, the phrase “lipid nanoparticle” or “nanoparticle” refers to a transfer vehicle comprising one or more lipids (e.g., cationic lipids, non-cationic lipids, and PEG-modified lipids). Preferably, the lipid nanoparticles are formulated to deliver one or more nucleic acid sequences to one or more target cells (e.g., CNS tissue and/or muscle). Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a nucleic acid sequence encapsulated therein to a target cell. Useful lipid nanoparticles for nucleic acid sequence comprise a cationic lipid to encapsulate and/or enhance the delivery of such nucleic acid sequence into the target cell that will act as a depot for protein production. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available. See, e.g., WO2014/089486, US 2018/0353616A1, and U.S. Pat. No. 8,853,377B2, which are incorporated by reference. In certain embodiments, LNP formulation is performed using routine procedures comprising cholesterol, ionizable lipid, helper lipid, PEG-lipid and polymer forming a lipid bilayer around encapsulated nucleic acid sequence (Kowalski et al., 2019, Mol. Ther. 27(4): 710-728). In some embodiments, LNP comprises a cationic lipids (i.e. N-[1-(2,3-dioleoyloxy)propyl]-N,N,N-trimethylammonium chloride (DOTMA), or 1,2-dioleoyl-3-trimethylammonium-propane (DOTAP)) with helper lipid DOPE. In some embodiments, LNP comprises an ionizable lipid Dlin-MC3-DMA ionizable lipids, or diketopiperazine-based ionizable lipids (cKK-E12). In some embodiments, polymer comprises a polyethyleneimine (PEI), or a poly(β-amino)esters (PBAEs). See, e.g., WO2014/089486, US 2018/0353616A1, US2013/0037977A1, WO2015/074085A1, U.S. Pat. No. 9,670,152B2, and U.S. Pat. No. 8,853,377B2, which are incorporated by reference. In certain embodiments, a lipid nanoparticle (LNP) comprises at least one core Y-G/A/R/K-Y/H-GNPA-T/R/H-RYFD-V/K (also referred as Y-X-X″-GNPA-X″-RYFD-X″, wherein X′ is G, A, R, K, X″ is Y or H, X″ is T, R or H, and X″″ is V or K; SEQ ID NO: 14) peptide. i.e., decorated surface with targeting peptide. In certain embodiments, a lipid nanoparticle (LNP) comprises at least one core YGYGNPATRYFDV (SEQ ID NO: 1) peptide. In certain embodiments, a lipid nanoparticle (LNP) comprises at least one core YAYGNPATRYFDV (SEQ ID NO: 2) peptide.

In certain embodiments, a composition, e.g., an rAAV having a modified capsid with at least one core Y-G/A/R/K-Y/H-GNPA-T/R/H-RYFD-V/K (SEQ ID NO: 14) peptide and optional linker sequences, a fusion polypeptide or protein, or a conjugate comprising a nanoparticle or chemical moiety, is useful for delivering a therapeutic to a patient in need thereof. In certain embodiments, a composition comprises an rAAV having a modified capsid with at least one core YGYGNPATRYFDV (SEQ ID NO: 1) peptide and optional linker sequences, a fusion polypeptide or protein, or a conjugate comprising a nanoparticle or chemical moiety, and is useful for delivering a therapeutic to a patient in need thereof, wherein the therapeutic is targeted for delivery in CNS (brain and/or spinal cord). In certain embodiments, a composition comprises an rAAV having a modified capsid with at least one core YAYGNPATRYFDV (SEQ ID NO: 2) peptide and optional linker sequences, a fusion polypeptide or protein, or a conjugate comprising a nanoparticle or chemical moiety, and is useful for delivering a therapeutic to a patient in need thereof, wherein the therapeutic is targeted for delivery in CNS (brain and/or spinal cord).

In certain embodiments, a composition comprises an rAAV having a modified capsid with at least one core YGYGNPATRYFDV (SEQ ID NO: 1) peptide and optional linker sequences, a fusion polypeptide or protein, or a conjugate comprising a nanoparticle or chemical moiety, and is useful for delivering a therapeutic to a patient in need thereof, wherein the therapeutic is targeted for delivery in CNS (brain and/or spinal cord), and de-targeted for liver, heart and/or lung cells. In certain embodiments, a composition comprises an rAAV having a modified capsid with at least one core YAYGNPATRYFDV (SEQ ID NO: 2) peptide and optional linker sequences, a fusion polypeptide or protein, or a conjugate comprising a nanoparticle or chemical moiety, and is useful for delivering a therapeutic to a patient in need thereof, wherein the therapeutic is targeted for delivery in CNS (brain and/or spinal cord), and de-targeted for liver, heart and/or lung cells.

In certain embodiments, a rAAV having a modified capsid as described herein may be delivered in a co-therapeutic regimen which further comprises one or more other active components. In certain embodiments, the regimen may involve co-administration of an immunomodulatory component. Such an immunomodulatory regimen may include, e.g., but are not limited to immunosuppressants such as, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, cyclosporin, tacrolimus, sirolimus, IFN—B, IFN-γ, an opioid, or TNF-α (tumor necrosis factor-alpha) binding agent. In certain embodiments, the immunosuppressive therapy may be started prior to the gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week, about 15 days, about 30 days, about 45 days, 60 days, or longer, as needed. Still other co-therapeutics may include, e.g., anti-IgG enzymes, which have been described as being useful for depleting anti-AAV antibodies (and thus may permit administration to patients testing above a threshold level of antibody for the selected AAV capsid), and/or delivery of anti-FcRN antibodies which is described, e.g., in WO 2021/257668, filed 23 Dec. 2021, (claiming priority to U.S. Provisional Patent Application No. 63/040,381, filed Jun. 17, 2020, U.S. Provisional Patent Application No. 62/135,998, filed Jan. 11, 2021, and U.S. Provisional Patent Application No. 63/152,085, filed Feb. 22, 2021) entitled “Compositions and Methods for Treatment of Gene Therapy Patients”, and/or one or more of a) a steroid or combination of steroids and/or (b) an IgG-cleaving enzyme, (c) an inhibitor of Fc-IgE binding; (d) an inhibitor of Fc-IgM binding; (e) an inhibitor of Fc-IgA binding; and/or (f) gamma interferon.

An antibody “Fc region” refers to the crystallizable fragment which is the region of an antibody which interacts with the cell surface receptors (Fc receptors). In one embodiment, the Fc region is a human IgG1 Fc. In one embodiment, the Fc region is a human IgG2 Fc. In one embodiment, the Fc region is a human IgG4 Fc. In one embodiment, the Fc region is an engineered Fc fragment. See, e.g., Lobner, Elisabeth, et al. “Engineered IgG1-Fc-one fragment to bind them all.” Immunological reviews 270.1 (2016): 113-131: Saxena, Abhishek, and Donghui Wu. “Advances in therapeutic Fc engineering-modulation of IgG-Associated effector functions and serum half-life.” Frontiers in immunology 7 (2016); Irani, Vashti, et al. “Molecular properties of human IgG subclasses and their implications for designing therapeutic monoclonal antibodies against infectious diseases.” Molecular immunology 67.2 (2015): 171-182; Rath, Timo, et al. “Fc-fusion proteins and FcRn: structural insights for longer-lasting and more effective therapeutics.” Critical reviews in biotechnology 35.2 (2015): 235-254; and Invivogen, IgG-Fc Engineering For Therapeutic Use, invivogen.com/docs/Insight200605.pdf, April 2006; each of which is incorporated by reference herein.

An antibody “hinge region” is a flexible amino acid portion of the heavy chains of IgG and IgA immunoglobulin classes, which links these two chains by disulfide bonds.

An “immunoglobulin molecule” is a protein containing the immunologically-active portions of an immunoglobulin heavy chain and immunoglobulin light chain covalently coupled together and capable of specifically combining with antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein.

An “immunoglobulin heavy chain” is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain.

An “immunoglobulin light chain” is a polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

“Neutralizing antibody titer” (NAb titer) a measurement of how much neutralizing antibody (e.g., anti-AAV NAb) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009, 199 (3): p. 381-390, which is incorporated by reference herein.

As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 10 provides the encoded amino acid sequence of the AAVhu68 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine (N-G) pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs. Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, 97%, 99%, up to about 100% deamidated at a referenced amino acid position, as compared to the predicted amino acid sequence at the reference amino acid position. Such percentages may be determined using 2D-gel, mass spectrometry techniques, or other suitable techniques.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected. See, e.g., WO 2019/168961, published Sep. 6, 2019, including Table G providing the deamidation pattern for AAV9 and WO 2020/160582, filed Sep. 7, 2018. See, also, e.g., WO 2020/223231, published Nov. 5, 2020 (rh91, including table with deamidation pattern), US Provisional Patent Application No. 63/065,616, filed Aug. 14, 2020, and U.S. Provisional Patent Application No. 63/109,734, filed Nov. 4, 2020 and International Patent Application No. PCT/US21/45945, filed Aug. 13, 2021, which are all incorporated herein by reference in its entirety.

The compositions described herein may be used in a regimen involving co-administration of other active agents. Any suitable method or route can be used to administer such other agents. Routes of administration include, for example, systemic, oral, intravenous, intraperitoneal, subcutaneous, or intramuscular administration. Optionally, the AAV compositions described herein may also be administered by one of these routes.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient: i.e., they cannot generate progeny virions but retain the ability to infect target cells. In one embodiment, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

A “recombinant AAV” or “rAAV” is a DNAse-resistant viral particle containing two elements, an AAV capsid and a vector genome containing at least non-AAV coding sequences packaged within the AAV capsid. In certain embodiments, the capsid contains about 60 proteins composed of vp1 proteins, vp2 proteins, and vp3 proteins, which self-assemble to form the capsid. Unless otherwise specified, “recombinant AAV” or “rAAV” may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

The term “nuclease-resistant” indicates that the AAV capsid has assembled around the expression cassette which is designed to deliver a transgene to a host cell and protects these packaged genomic sequences from degradation (digestion) during nuclease incubation steps designed to remove contaminating nucleic acids which may be present from the production process.

As used herein, the term “host cell” may refer to the packaging cell line in which the rAAV is produced from the plasmid. In the alternative, the term “host cell” may refer to the target cell in which expression of the transgene is desired.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s) (i.e., transgene(s)), and an AAV 3′ ITR. In certain embodiments, the ITRs are from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein. In one example, a “vector genome” contains, at a minimum, from 5′ to 3′, a vector-specific sequence, a nucleic acid sequence encoding protein of interest operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids.

As used herein, “operably linked” sequences include both expression control sequences that are contiguous with the gene of interest and expression control sequences that act in trans or at a distance to control the gene of interest.

In certain embodiments, non-viral genetic elements used in manufacture of a rAAV, will be referred to as vectors (e.g., production vectors). In certain embodiments, these vectors are plasmids, but the use of other suitable genetic elements is contemplated. Such production plasmids may encode sequences expressed during rAAV production, e.g., AAV capsid or rep proteins required for production of a rAAV, which are not packaged into the rAAV. Alternatively, such a production plasmid may carry the vector genome which is packaged into the rAAV.

As used herein, a “parental capsid” refers to a non-mutated or a non-modified capsid selected from parvovirus or other viruses (e.g., AAV, adenovirus, HSV, RSV, etc.). In certain embodiments, the parental capsid includes any naturally occurring AAV capsids comprising a wild-type genome encoding for capsid proteins (i.e., vp proteins), wherein the capsid proteins direct the AAV transduction and/or tissue-specific tropism. In some embodiments, the parent capsid is selected from AAV which natively targets CNS. In other embodiments, the parental capsid is selected from AAV which do not natively target CNS.

As used herein, the terms “target cell” and “target tissue” can refer to any cell or tissue which is intended to be transduced by the subject AAV vector. The term may refer to any one or more of muscle, liver, lung, airway epithelium, central nervous system, neurons, eye (ocular cells), or heart. In one embodiment, the target tissue is brain. In certain embodiments, the target cell is one or more cell type of the CNS (e.g., brain cell), including but not limited to astrocytes, neurons, glial cells, ependymal cells, and cells of the choroid plexus.

As used herein, a “variant capsid” or a “variant AAV” or “variant AAV capsid” refers to a modified capsid or a mutated capsid, wherein the capsid protein comprises an insertion of a tissue-specific targeting peptide.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell. Typically, such an expression cassette can be used for generating a viral vector and contains the coding sequence for the gene product described herein flanked by packaging signals of the viral genome and other expression control sequences such as those described herein. In certain embodiments, a vector genome may contain two or more expression cassettes.

The term “translation” in the context of the present invention relates to a process at the ribosome, wherein an mRNA strand controls the assembly of an amino acid sequence to generate a protein or a peptide.

The term “expression” is used herein in its broadest meaning and comprises the production of RNA or of RNA and protein. Expression may be transient or may be stable.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13): 2682-2690 (1999).

In certain embodiments, an effective amount may be determined based on an animal model, rather than a human patient.

As described above, the term “about” when used to modify a numerical value means a variation of ±10%, (±10%, e.g., ±1, ±2, ±3, ±4, ±5, ±6, ±7, ±8, ±9, ±10, or values therebetween) from the reference given, unless otherwise specified.

In certain instances, the term “E+#” or the term “e+#” is used to reference an exponent. For example, “5E10” or “5e10” is 5×10¹⁰. These terms may be used interchangeably.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following examples are illustrative only and are not a limitation on the invention described herein.

The Adeno-Associated Virus (AAV) capsid can safely deliver gene correction to many tissues following intravenous (IV) delivery. However, even so-called “neurotropic” natural serotypes show poor efficiency of gene transfer to the central nervous system due to the blood-brain barrier (BBB). Here we present a novel AAV library strategy to discover peptides capable of directing AAV9 across the BBB. We first identified hundreds of candidate peptides with the potential to interact with proteins residing on brain capillaries. We produced a library of AAV9 variants displaying these peptides on the capsid surface in a variety of structural contexts. Next-generation sequencing- (NGS-) analysis of capsid genes from brain tissue revealed remarkable brain-transduction activity of a variant, “YGY”, that harbors a 13 amino-acid insertion (YGYGNPATRYFDV; SEQ ID NO: 1) at HVRVIII. Next, we deployed an NGS-based optimization pipeline to simultaneously improve vector production yield and BBB-crossing in AAV9-YGY. Optimized AAV9-YGY variants show superior brain transduction from IV delivery as compared to a benchmark engineered capsid, AAV9-PHP.B, in C57BL/6J mice. Unlike AAV9-PHP.B, however, YGY variants also function in strains not permissive to AAV9-PHP.B, such as Balb/c. We discuss the implications of this work for targeting the AAV capsid to tissue-specific receptors and contrast it to more typical, unbiased, large-library screening approaches which often prove ineffective in large animal models.

### Example 1. Primary Screen in Mice

It has been shown that small peptide insertions into flexible loop on the surface of the AAV capsid can mediate interactions with new cellular receptors. In one case discovered at CalTech (AAV9-PHP.B), a seven amino acid peptide inserted into the HVRVIII loop on AAV9 mediates interaction with Ly6a, a GPI-anchored receptor on the brain vasculature of some mouse strains. This interaction drives transport of AAV9-PHP.B across the BBB, resulting in ˜50-fold higher transduction of brain cells than AAV9. In this work, we search for peptide inserts that can bind cell membrane targets on the BBB and thus have the potential to drive the AAV9 capsid across the BBB.

We sought to solve the AAV-BBB problem by first surveying the available academic and patent literature for peptide sequences that may have the potential to interact with the vascular cells in the brain. We found the following sources of these peptides:
Published results of phage-display experiments in which phage display libraries were panned against primary brain endothelial cells;
Natural ligand peptides to known BBB-resident membrane proteins;
CDRs of antibodies targeted to BBB-resident membrane proteins;
Viral coat proteins of flaviviruses that cause encephalitis; and
Bacterial toxins that have cell-binding activities directed at GPI anchorages.

We generated a library of AAV9 insertion mutants containing hundreds of peptides from these sources, all inserted individually at the HVRVIII locus (between position 588 and 589, based on the numbering of the amino acid sequence of AAV9 capsid of SEQ ID NO: 9). See also, for reference an alignment of the HVRIII region in various AAV capsid protein fragments. FIG. 13 shows region of the alignment of the amino acid sequences of the various AAV capsid proteins of AA9 (amino acids 566 to 615 of AAV9 capsid; SEQ ID NO: 30), AAV8 (amino acids 565 to 614 of AAV8 capsid; SEQ ID NO: 31), AAV7 (amino acids 567 to 616 of AAV7; SEQ ID NO: 32), AAV6 (amino acids 550 to 599 of AAV6 capsid; SEQ ID NO: 33), AAV5 (amino acids 556 to 605 of AAV5; SEQ ID NO: 34), AAV4 (amino acids 558 to 607 of AAV4 capsid; SEQ ID NO: 35), AAV3B (amino acids 564 to 613 of AAV3B capsid; SEQ ID NO: 36), AAV2 (amino acids 566 to 615 of AAV2 capsid; SEQ ID NO: 37), and AAV1 (amino acids 566 to 615 of AAV1 capsid; SEQ ID NO: 38), which is focused on the region HVRVIII in which the targeting peptide may be inserted (based on structure analysis).

Each peptide was typically present in the library in multiple forms that differed by 1) length of peptide inserted 2) presence of flexible GSG or GG linker sequences on both sides of the peptide. Peptides were also encoded using multiple synonymous codons so that we could independently observe replicate activities in the screen.

As a control a PHP.B peptide was included as well (positive control for C57/BL6 and negative control for Balb/c & NHP). Each peptide was encoded in multiple ways (with and without a linker, and in several synonymous DNA sequences).

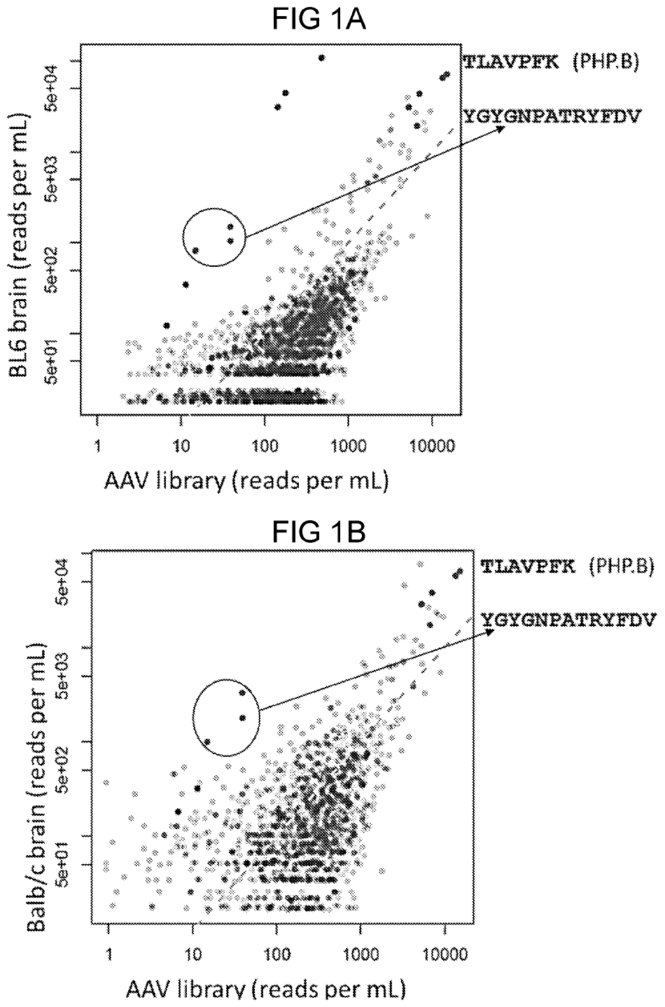

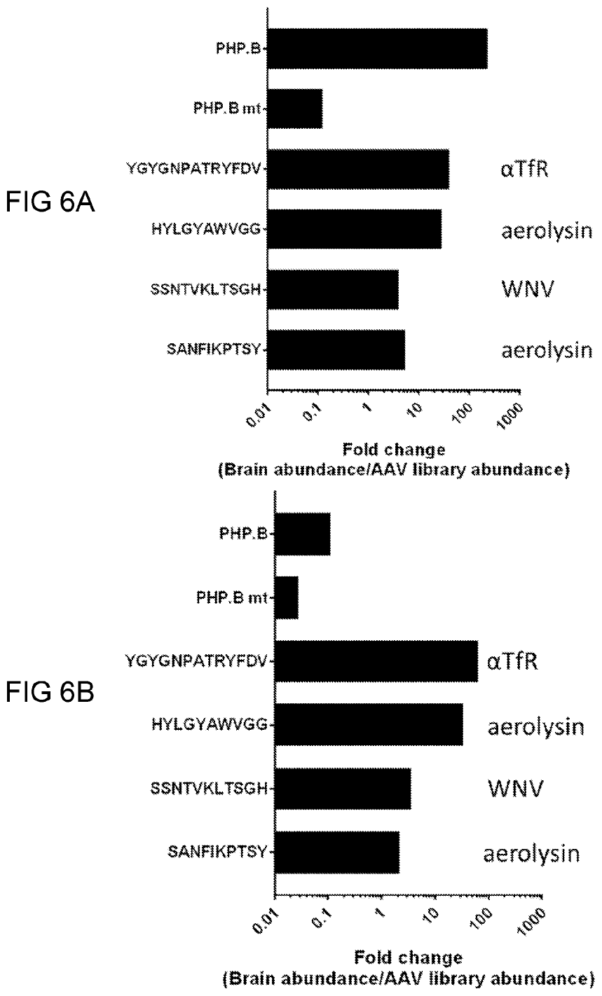

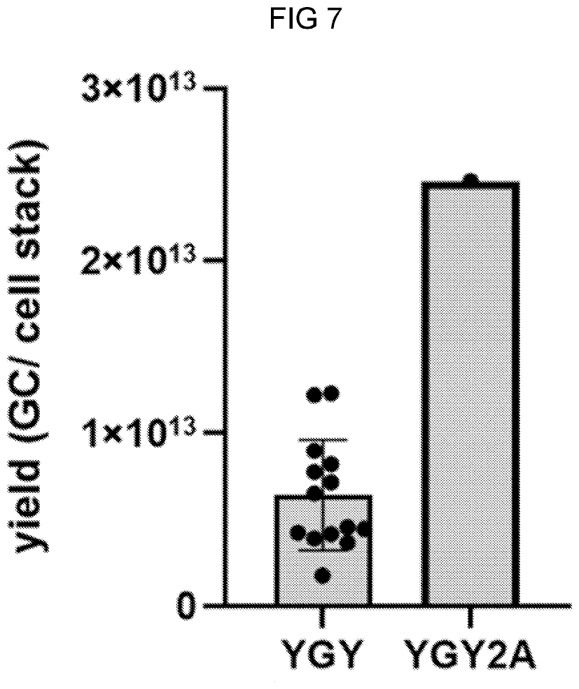

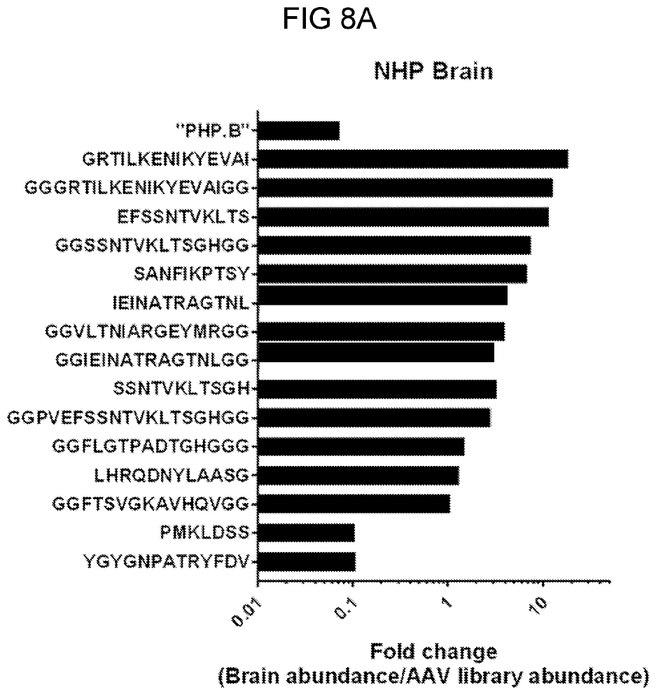

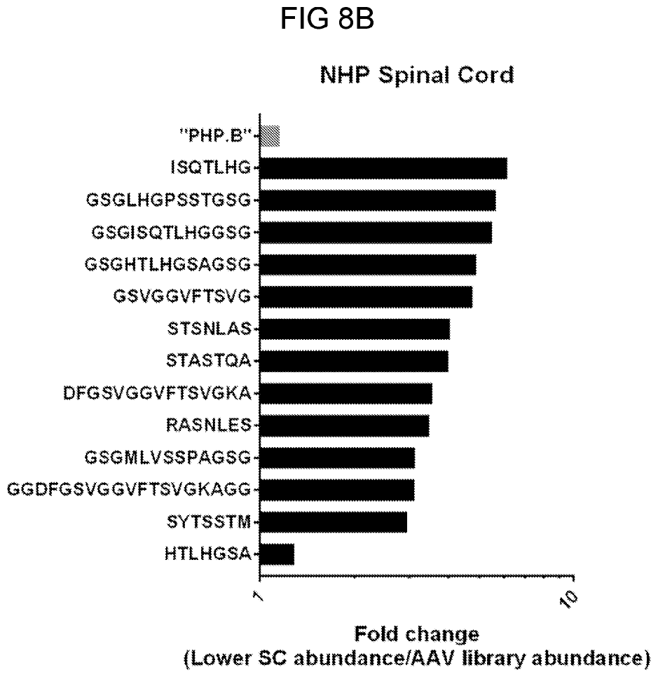

The engineering strategy for AAV BBB-specific capsid selection, via a screening technique of a BBB Candidate mini library comprising about 10³ variants of peptides sourced from literature and patents (figure not shown). A BBB Candidate min-library was generated (˜10³), comprising hundreds of peptides. We injected this library at high-dose intravenously (IV) to 2 mouse strains and to one non-human primate. After a 2-3 week in-life period, the animals were necropsied, and tissues were collected. We extracted the DNA genomes of AAV vectors from CNS and other tissues, and subjected these to next-generation sequencing (NGS). The vector variants encapsidate their own capsid gene variant, allowing us to track capsid activity through the relative abundance of the capsid gene variant in the tissue of interest. We scored the BBB activity (“enrichment score”) of each variant in the library by calculating its abundance in the CNS normalized to its abundance in the injected library mixture. Enrichment scores were examined over the injected library in C57/BL6 mice (FIGS. 1A, 6A), Balb/c mice (FIGS. 1B, 6B) and NHP brain (FIGS. 8A and 8B).

In mouse study, top brain enriched HVRVIII insertions in C57/BL6 mice (FIG. 1A) and Balb/c mice (FIG. 1B) were: TLAVPFK (SEQ ID NO: 11) (PHP.B), YGYGNPATRYFDV (SEQ ID NO: 1), and HYLGYAWVGG (SEQ ID NO: 15), EFSSNTVKLTS (SEQ ID NO: 16), and SANFIKPTSY (SEQ ID NO: 17). Positive control PHP.B comes up 3 times independently as the most enriched hit. Three of the PHP.B peptides with synonymous codons are independently enriched. Several other peptides are also enriched in brain. FIGS. 6A and 6B show the enrichment scores for the best mouse brain hits in the screen, with referenced peptides (FIG. 6A for C57BL/6J mice; and FIG. 6B for Balb/c mice).

### Example 2. Secondary Validation of Hits in Mice

Next, we followed up the primary screen in mice by generating GFP reporter vectors for several of the hit capsids. The vectors were injected at high dose IV to C57BL/6J mice. 2 weeks later, we necropsied the mice and collected GFP images of brain sections (data not shown). All of the hit vectors tested in the GFP study were de-localized from the liver, as evident from liver GFP staining (data not shown).

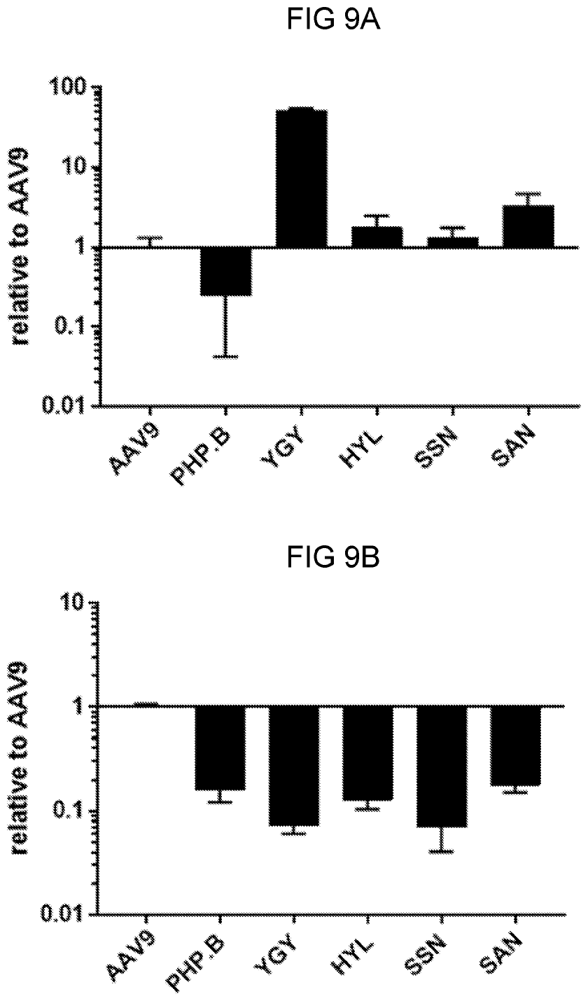

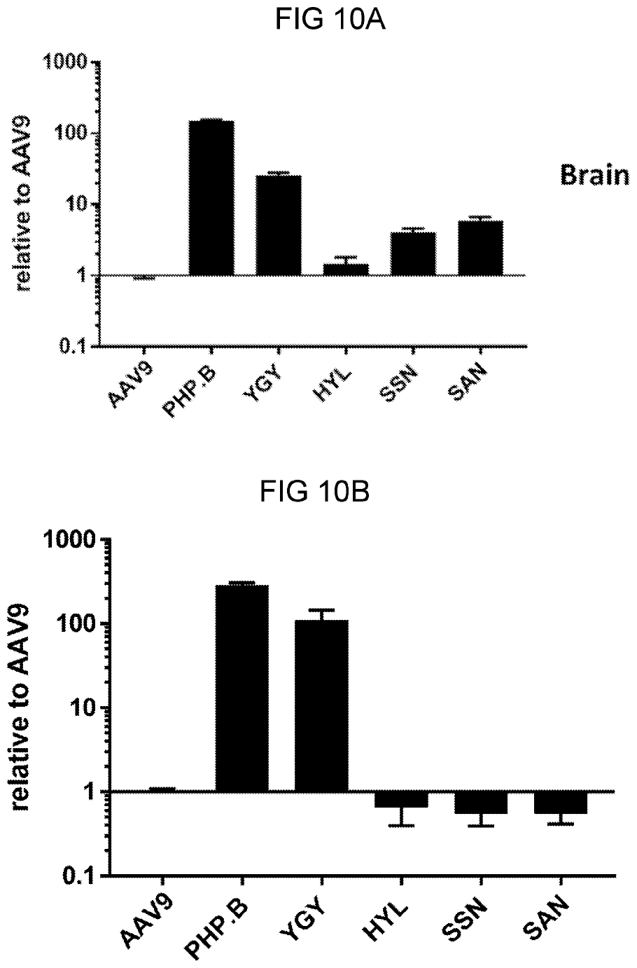

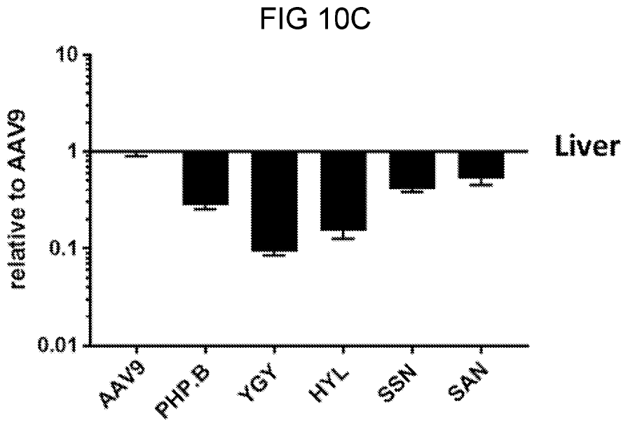

We confirmed these BBB-crossing and brain-localizing activities in a barcoded vector study. Briefly, each capsid was used to individually produce vector containing a GFP reporter gene with a unique DNA barcode included. The barcoded capsid preps were mixed in equal proportions, and injected into C57BL/6J or Balb/c mice (FIGS. 9A-9B, and 10A-10C). At the conclusion of in-life period, mice were sacrificed, necropsied and tissues were subjected to NGS sequencing to count the abundance of each barcode among the vector genomes extracted from the tissue. The results confirm brain localization of vector genomes for all the hit capsids identified in the primary screen. In Balb/c mice, the secondary validation screen showed brain targeting for all hit sequences discovered in primary screen, especially “YGY” (FIG. 9A). In C576BL/6 mice the secondary validation screen showed brain targeting for all hit sequences discovered in primary screen (FIG. 10A). In both, Balb/c and C57BL/6 mice, liver de-targeting for all hit sequences, relative to AVA9 was consistent with affinity for brain vasculature (FIGS. 9B and 10C). A secondary validation was performed of the transduction levels of top performing peptide hits in AAV capsid comprising GFP reporter transgene in cortex, hippocampus, thalamus, cerebellum, and liver (microscopy images not shown). Briefly, AAV-PHP.B (1×10¹²) and AAV9-YGY (7×10¹¹) showed lower GFP expression levels in liver tissue in comparison to AAV9 (1×10¹²) (i.e., liver de-targeting). AAV-PHP.B (1×10¹²) showed modestly higher and AAV9-YGY (7×10¹¹) showed slightly higher GFP expression levels in cerebellum tissue in comparison to AAV9 (1×10¹²). AAV-PHP.B (1×10¹²) showed and AAV9-YGY (7×10¹¹) showed higher GFP expression levels in hippocampus tissue in comparison to AAV9. AAV-PHP.B (1×10¹²) showed and AAV9-YGY (7×10¹¹) showed higher GFP expression levels in cortex tissue in comparison to AAV9 (1×10¹²). These results confirmed that YGY is an efficient BBB-crossing capsid. Furthermore, it was shown that cerebellum is an exception (lower expression levels observed in comparison to thalamus, hippocampus and cortex for AAV-YGY and AAV-PHP.B), demonstrating BBB heterogeneity.

### Example 3. Hit Optimization

In this study, we produced vector for optimization libraries comprising all possible single point mutations in the insert sequence, and truncations of the insert sequence. For the truncation-based library, a mini library of about 150 truncated versions of “YGY” core peptides was used, which included all possible truncations (N- and C-terminal) of the 13 amino acid insert. Vector libraries, truncation-based and single-point mutation-based, were then injected into mice. After a 2 week in-life period, mice were necropsied, and brain tissues were collected. We extracted capsid mRNA from brain tissue, and subjected these to next-generation sequencing (NGS). Each variant was scored relative to the original “YGY” core peptide. Yield scores and BBB scores were examined.

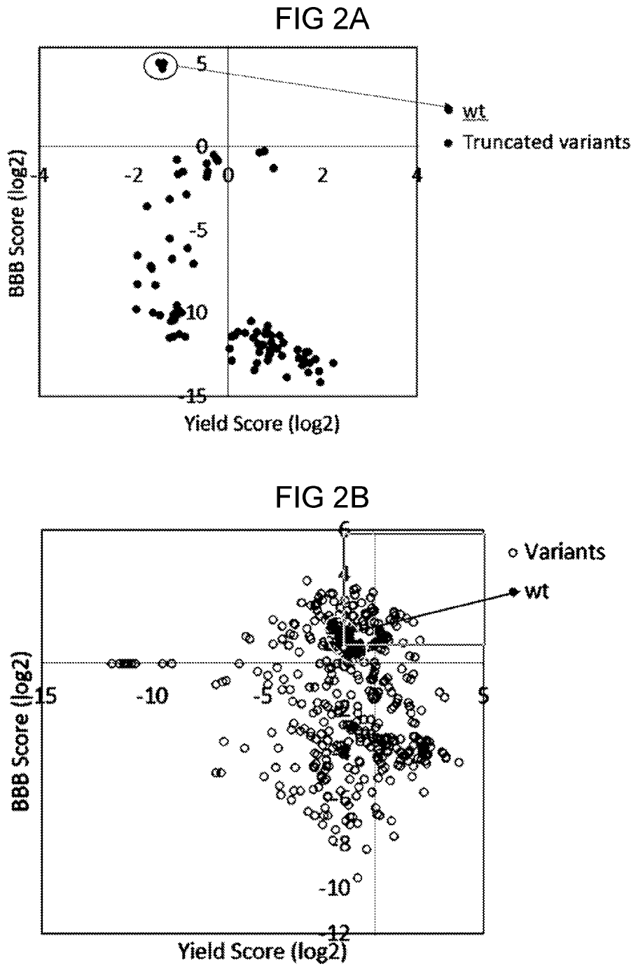

The examined scores of truncation-based library showed that while almost all “YGY” truncations improved yield, all impaired brain localization (FIG. 2A). However, the examined scores of comprehensive single site “YGY” core peptide mutation library showed that some “YGY” core peptide variants may improve yield level in comparison to of those observed in AAV9, while also retaining BBB activity (FIG. 2B). In examination of “YGY” (SEQ ID NO: 1) original core peptide, the yield was down 90% in comparison to that of native AAV9, when examined in mega scale, or 80% when examined in a small scale. The selected top 7 candidates of “YGY” core peptide variants, which improved yield to AAV9 and retained BBB activity are shown in Table 1 below.

TABLE 1

| | Amino acid position | | | | | | | | | | | | | SEQ |
|---|---|---|---|---|---|---|---|---|---|---|---|---|---|---|
| Name | 1 | 2 | 3 | 4 | 5 | 6 | 7 | 8 | 9 | 10 | 11 | 12 | 13 | ID NOs |
| YGY | Y | G | Y | G | N | P | A | T | R | Y | F | D | V | 1 |
| YGY2A | Y | A | Y | G | N | P | A | T | R | Y | F | D | V | 2 |
| YGY2K | Y | K | Y | G | N | P | A | T | R | Y | F | D | V | 3 |
| YGY2R | Y | R | Y | G | N | P | A | T | R | Y | F | D | V | 4 |
| YGY3H | Y | G | H | G | N | P | A | T | R | Y | F | D | V | 5 |
| YGY8R | Y | G | Y | G | N | P | A | R | R | Y | F | D | V | 6 |
| YGY8H | Y | G | Y | G | N | P | A | H | R | Y | F | D | V | 7 |
| YGY13K | Y | G | Y | G | N | P | A | T | R | Y | F | D | K | 8 |

Table 2 below shows summary of the yield score, brain mRNA score, and brain DNA score as measured for the above-described top 7 “YGY” core peptide variants.

TABLE 2

| | | Yield | Brain mRNA | Brain DNA |
|---|---|---|---|---|
| AA | AA position | Score | Score | score |
| K | 2 | 10.4 | 0.9 | 0.9 |
| R | 2 | 7.0 | 1.5 | 1.7 |
| K | 13 | 6.1 | 1.0 | 0.8 |
| R | 8 | 4.7 | 1.2 | 2.0 |
| H | 3 | 4.6 | 1.5 | 1.5 |
| A | 2 | 2.4 | 4.2 | 2.2 |
| H | 8 | 2.3 | 1.8 | 2.2 |
| Original YGY | | 1.0 | 1.0 | 1.0 |

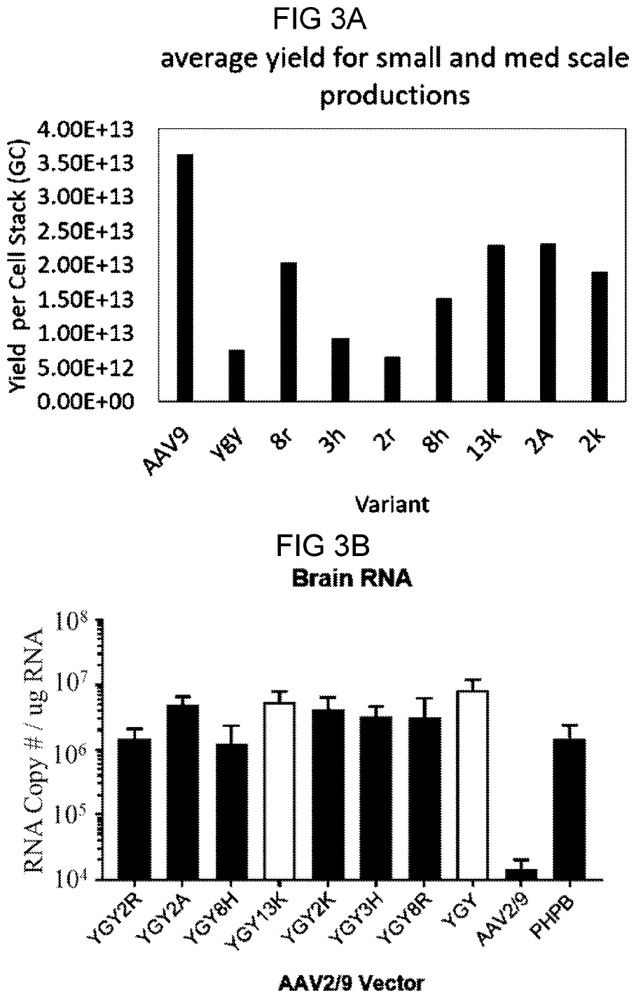

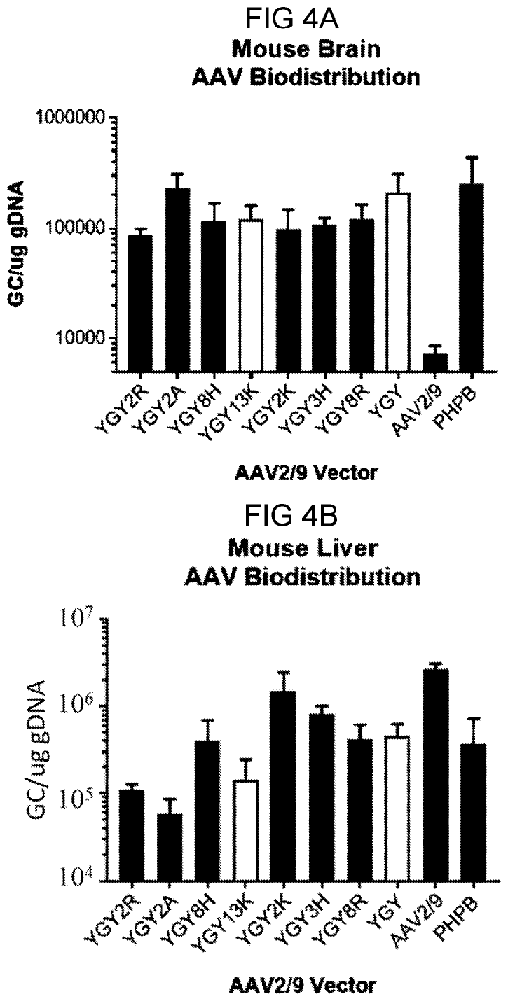

Next, we examined the yield of a small and medium scale productions of the above-mentioned top 7 “YGY” core peptide variant inserts in AAV9-variant capsids comprising a GFP transgene, and compared the resulting yields to those of native AAV9 and AAV9-variant comprising original “YGY” core peptide insert (FIG. 3A). The results showed that YGY2A insert and YGY13K insert improved yield of AAV9 up to 65% in comparison to the original “YGY” core peptide insert. Furthermore, we examined function of AAV-variants comprising “YGY” top 7 performing peptide inserts in mice (transgene: GFP; mice: C57BL/6J). For this study, native AAV9 or specified AAV9-variants were delivered intravenously at a dose of 1×10¹² GC. FIG. 4A shows native AAV9 or AAV9-variant biodistribution in mouse (C57BL/6J) brain, plotted as GC/μg DNA. FIG. 4B shows native AAV9 or AAV9-variant biodistribution in mouse (C57BL/6J) liver, plotted as GC/μg DNA. The results showed that all of the top 7 “YGY” core peptide variants retained strong brain cell targeting. Additionally, when delivered systemically (i.e., intravenously), all of the AAV9-variants showed liver de-targeting, as shown by biodistribution, though to varying degrees. FIG. 3B shows a preliminary transduction test with various AAV9-GFP vectors in mouse (C57BL/6J) brain, wherein results of the transduction test were plotted as a ratio of mRNA copy number over micro-gram total mRNA. Some of the variant AAV9-inserts, including the original “YGY” core peptide, showed superior transduction efficacy to that of AAV9-PHP.B. FIG. GFP expression was examined in cells of collected brain and liver tissues from mice (C57BL/6J) following intravenous delivery of AAV9, AAV9-PHP.B, AAV9-YGY, and AAV9-YGY-variants (data not shown). Briefly, AAV9-YGY2R yielded in GFP expression levels similar in comparison to AAV9-PHP.B and higher in comparison to AAV9 in brain tissue. AAV9-YGY2R yielded in GFP expression levels slightly higher in comparison to AAV9-PHP.B and significantly lower in comparison to AAV9 in liver tissue. AAV9-YGY2A (comprising peptide core YAYGNPATRYFDV; SEQ ID NO: 2) yielded GFP expression levels higher in comparison to AAV9-PHP.B and significantly higher in comparison to AAV9 in brain tissue.

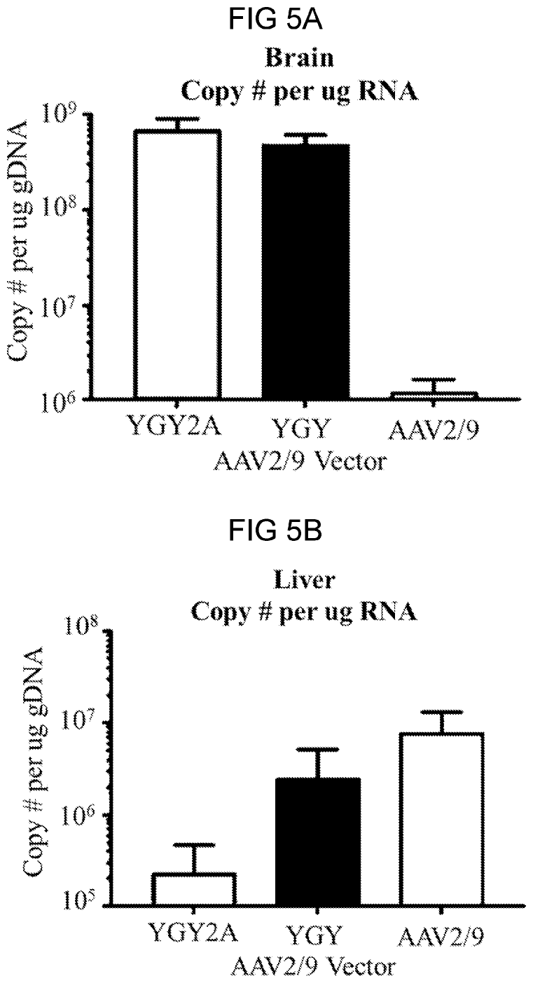

AAV9-YGY2A yielded GFP expression levels similar in comparison to AAV9-PHP.B and significantly lower in comparison to AAV9 in liver tissue. AAV9-YGY8H yielded GFP expression levels similar in comparison to AAV9-PHP.B and higher in comparison to AAV9 in brain tissue. AAV9-YGY8H yielded GFP expression levels higher in comparison to AAV9-PHP.B and similar in comparison to AAV9 in liver tissue. AAV9-YGY13K yielded GFP expression levels slightly higher in comparison to AAV9-PHP.B and significantly higher in comparison to AAV9 in brain tissue. AAV9-YGY13K yielded GFP expression levels similar in comparison to AAV9-PHP.B and significantly lower in comparison to AAV9 in liver tissue. AAV9-YGY2K yielded GFP expression levels similar in comparison to AAV9-PHP.B and slightly higher in comparison to AAV9 in brain tissue. AAV9-YGY2K yielded GFP expression levels higher in comparison to AAV9-PHP.B and similar in comparison to AAV9 in liver tissue. AAV9-YGY3H yielded GFP expression levels slightly lower in comparison to AAV9-PHP.B and similar in comparison to AAV9 in brain tissue. AAV9-YGY3H yielded GFP expression levels slightly higher in comparison to AAV9-PHP.B and slightly lower in comparison to AAV9 in liver tissue. AAV9-YGY8R yielded GFP expression levels similar in comparison to AAV9-PHP.B and slightly higher in comparison to AAV9 in brain tissue. AAV9-YGY8R yielded GFP expression levels similar in comparison to AAV9-PHP.B and lower in comparison to AAV9 in liver tissue. AAV9-YGY yielded GFP expression levels slightly higher in comparison to AAV9-PHP.B and significantly higher in comparison to AAV9 in brain tissue. AAV9-YGY yielded in GFP expression levels higher in comparison to AAV9-PHP.B and similar in comparison to AAV9 in liver tissue. GFP expression was in cells from various brain regions (i.e., cortex, thalamus, hippocampus, cerebellum) of the collected tissue from mice (C57BL/6J) following intravenous delivery of AAV9, AAV9-PHP.B, AAV9-YGY, and AAV9-YGY2A variant (data not shown). Briefly, AAV9-YGY and AAV9-YGY2A yielded in slightly higher GFP expression levels in comparison to AAV9-PHP.B and modest-significantly higher GFP expression levels in comparison to AAV9 in hippocampus and thalamus. AAV9-YGY and AAV9-YGY2A yielded in similar GFP expression levels in comparison to AV9-PHP.B and modest-significantly higher GFP expression levels in comparison to AAV9 in cortex. AAV9-YGY and AAV9-YGY2A yielded in slightly lower GFP expression levels in comparison to AV9-PHP.B and similar GFP expression levels in comparison to AAV9 in cerebellum. The results showed that “YGY” core peptide-comprising AAV9 vector variants have superior transduction efficacy in comparison to that of AAV9-PHP.B in all examined regions of the brain, except for cerebellum. Similar results were observed when AAV9 and AAV9-variant vectors were examined in Balb/c mice. FIG. 5A shows a transduction test with AA9V-GFP vectors in mouse (Balb/c) brain, wherein results of the transduction test were plotted as a ratio of mRNA copy number over micro-gram total mRNA. FIG. 5B shows a transduction test with GFP vectors in mouse liver, wherein results of the transduction test were plotted as a ratio of mRNA copy number over micro-gram total mRNA. GFP expression was examined in cells from various brain regions (i.e., cortex, thalamus, hippocampus) of the collected tissue from mice (Balb/c) following intravenous delivery of AAV9, AAV9-YGY, and AAV9-YGY2A (data not shown). Briefly, AAV9-YGY and AAV9-YGY2A yielded in higher expression levels in cortex and significantly higher expression levels in thalamus and hippocampus in comparison to AAV9.

Here we identified a diverse set of bioactive, BBB-targeting (brain cell targeting) peptides and demonstrated the potential to import these peptides into the AAV context to generate mini-libraries enriched in BBB interaction capability. We identified a novel insert “YGY” which can be used in an AAV vector that crosses the BBB efficiently in multiple mouse strains. Additionally, we deployed an NGS-based optimization pipeline to further enhance yield characteristics and BBB targeting activity of this primary hit vector.

### Example 4. Vector Biodistribution in Various Tissues

In this study, C57BL/6 mice (5 mice per group) were administered with 1×10¹² (1E12) GC (GC/mouse) of AAV2/9-YGY, AAV2/9-YGY2A, or AAV2/9. Mice were examined for 14 days (14-day in-life), following which mice were euthanized, and tissues were collected for vector biodistribution analysis.

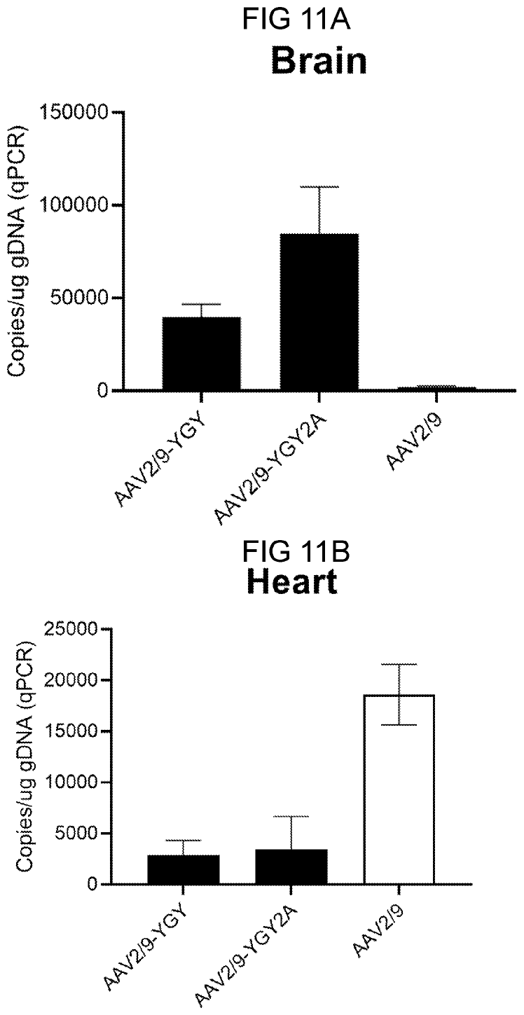

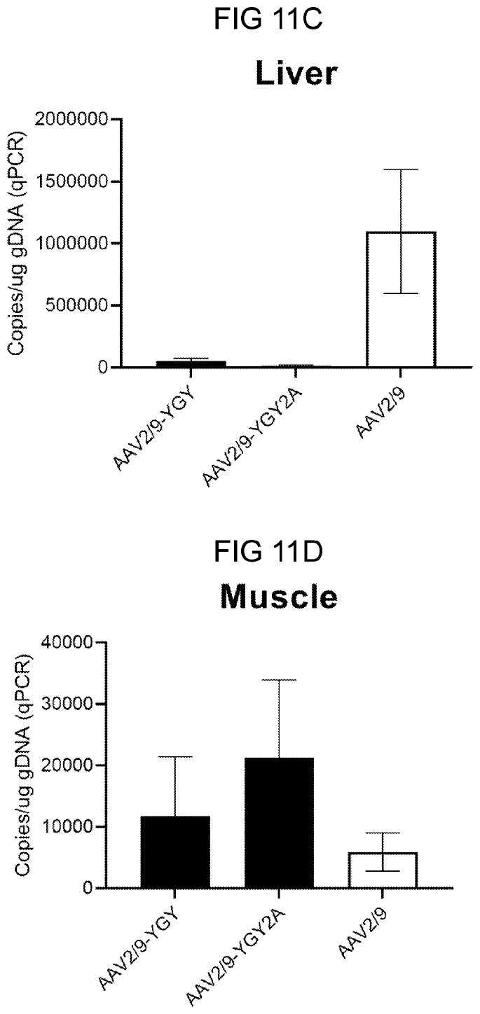

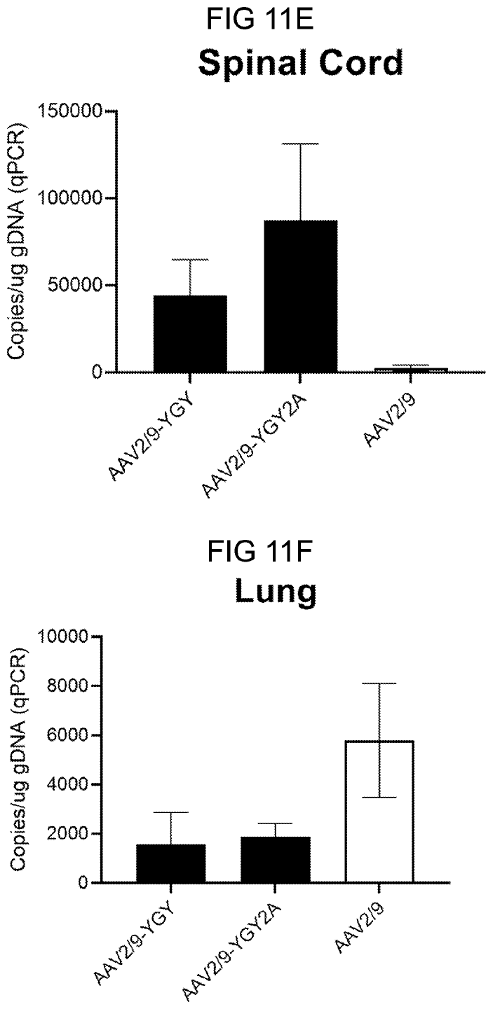

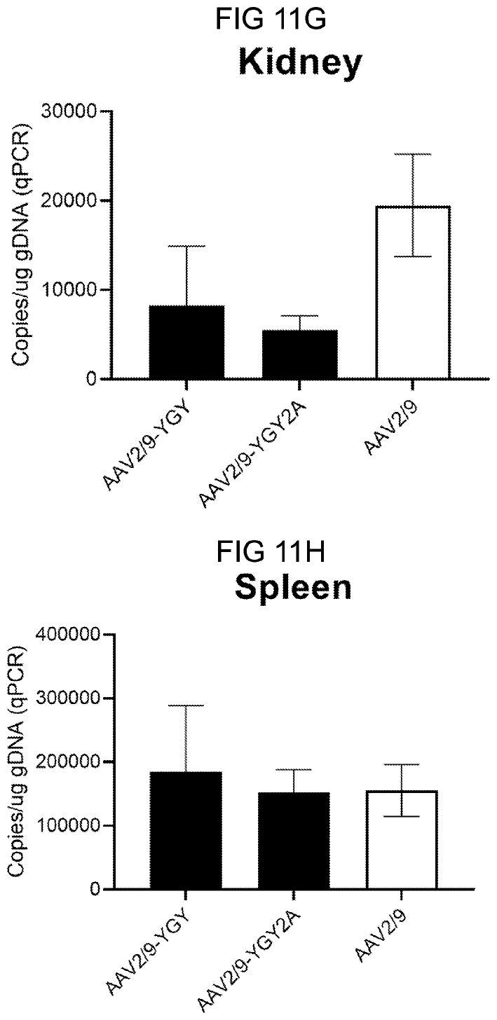

FIGS. 11A to 11H show biodistribution in various tissues (plotted as Copies/μg gDNA (qPCR)) of AAV2/9-YGY and AAV2/9-YGY2A vectors in C57BL/6J mice, compared to AAV9 vectors. FIG. 11A shows biodistribution in brain (plotted as Copies/μg gDNA (qPCR)) of AAV2/9-YGY and AAV2/9-YGY2A vectors in C57BL/6J mice, compared to AAV9 vectors. FIG. 11B shows biodistribution in heart (plotted as Copies/μg gDNA (qPCR)) of AAV2/9-YGY and AAV2/9-YGY2A vectors in C57BL/6J mice, compared to AAV9 vectors. FIG. 11C shows biodistribution in liver (plotted as Copies/μg gDNA (qPCR)) of AAV2/9-YGY and AAV2/9-YGY2A vectors in C57BL/6J mice, compared to AAV9 vectors. FIG. 11D shows biodistribution in muscle (plotted as Copies/μg gDNA (qPCR)) of AAV2/9-YGY and AAV2/9-YGY2A vectors in C57BL/6J mice, compared to AAV9 vectors. FIG. 11E shows biodistribution in spinal cord (plotted as Copies/μg gDNA (qPCR)) of AAV2/9-YGY and AAV2/9-YGY2A vectors in C57BL/6J mice, compared to AAV9 vectors. FIG. 11F shows biodistribution in lung (plotted as Copies/μg gDNA (qPCR)) of AAV2/9-YGY and AAV2/9-YGY2A vectors in C57BL/6J mice, compared to AAV9 vectors. FIG. 11G shows biodistribution in kidney (plotted as Copies/μg gDNA (qPCR)) of AAV2/9-YGY and AAV2/9-YGY2A vectors in C57BL/6J mice, compared to AAV9 vectors. FIG. 11H shows biodistribution in spleen (plotted as Copies/μg gDNA (qPCR)) of AAV2/9-YGY and AAV2/9-YGY2A vectors in C57BL/6J mice, compared to AAV9 vectors.

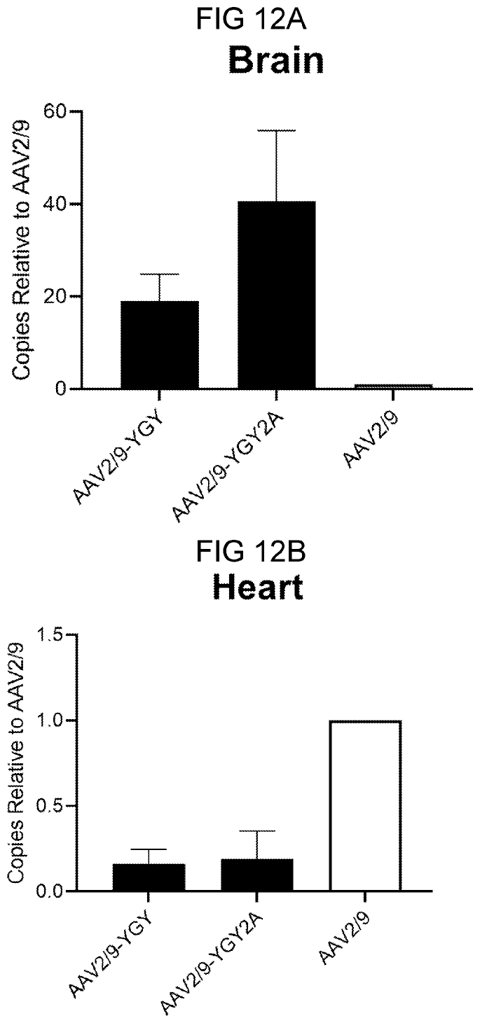

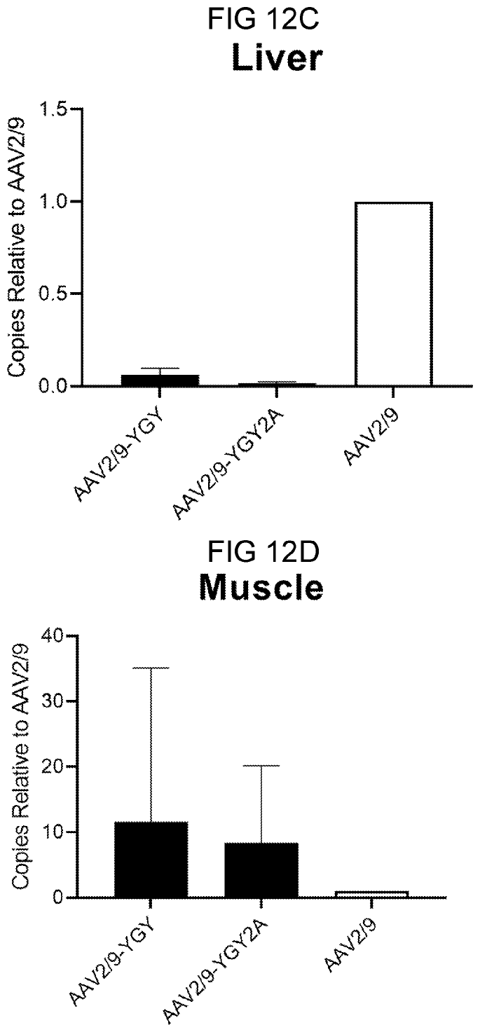

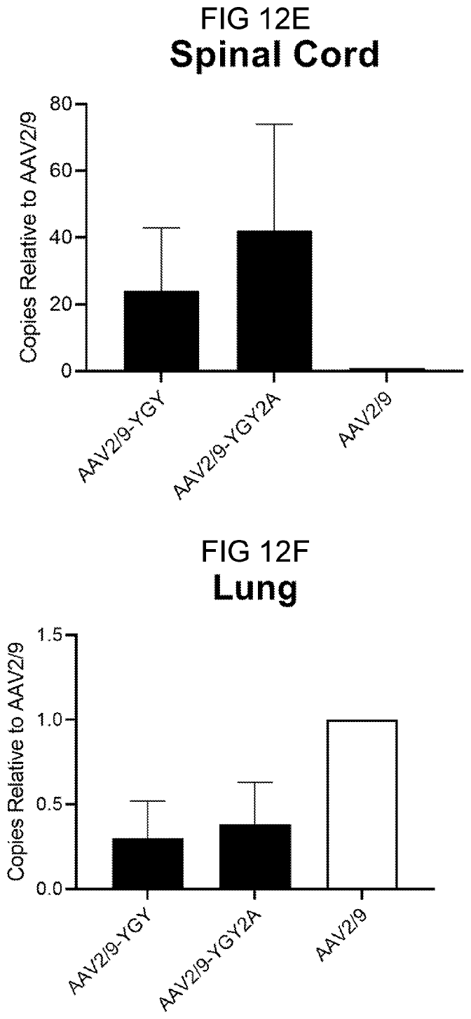

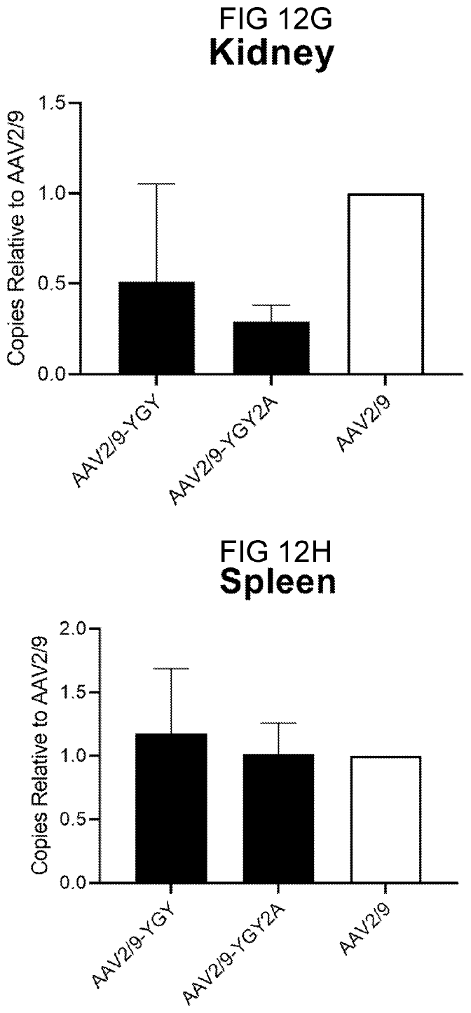

FIGS. 12A to 12H show biodistribution in various tissues (plotted as Copies Relative to AAV2/9) of AAV2/9-YGY and AAV2/9-YGY2A vectors in C57BL/6J mice, compared to AAV9 vectors. FIG. 12A shows biodistribution in brain (plotted as Copies Relative to AAV2/9) of AAV2/9-YGY and AAV2/9-YGY2A vectors in C57BL/6J mice, compared to AAV9 vectors. FIG. 12B shows biodistribution in heart (plotted as Copies Relative to AAV2/9) of AAV2/9-YGY and AAV2/9-YGY2A vectors in C57BL/6J mice, compared to AAV9 vectors. FIG. 12C shows biodistribution in liver (plotted as Copies Relative to AAV2/9) of AAV2/9-YGY and AAV2/9-YGY2A vectors in C57BL/6J mice, compared to AAV9 vectors. FIG. 12D shows biodistribution in muscle (plotted as Copies Relative to AAV2/9) of AAV2/9-YGY and AAV2/9-YGY2A vectors in C57BL/6J mice, compared to AAV9 vectors. FIG. 12E shows biodistribution in spinal cord (plotted as Copies Relative to AAV2/9) of AAV2/9-YGY and AAV2/9-YGY2A vectors in C57BL/6J mice, compared to AAV9 vectors. FIG. 12F shows biodistribution in lung (plotted as Copies Relative to AAV2/9) of AAV2/9-YGY and AAV2/9-YGY2A vectors in C57BL/6J mice, compared to AAV9 vectors. FIG. 12G shows biodistribution in kidney (plotted as Copies Relative to AAV2/9) of AAV2/9-YGY and AAV2/9-YGY2A vectors in C57BL/6J mice, compared to AAV9 vectors. FIG. 12H shows biodistribution in spleen (plotted as Copies Relative to AAV2/9) of AAV2/9-YGY and AAV2/9-YGY2A vectors in C57BL/6J mice, compared to AAV9 vectors.

These data confirm that AAV9-YGY and AAV9-YGY2A have a large transduction advantage in the brain over AAV9 and are liver de-targeted, in comparison to AAV9. Additionally, these data also show that AAV9-YGY and AAV9-YGY2A have a large transduction advantage in the spinal cord, while having a modest transduction advantage in muscle, and are de-targeting in heart and lung, in comparison to AAV9.

All documents cited in this specification are incorporated herein by reference. U.S. Provisional Patent Application No. 63/178,881, filed Apr. 23, 2021 is incorporated herein by reference in its entirety. The sequence listing filed herewith named “21-9637PCT_Sequences_ST25” and the sequences and text therein are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated virus (AAV) particle (rAAV) comprising:
(a) an AAV capsid comprising VP1 proteins, VP2 proteins and VP3 proteins, wherein the VP3 proteins has comprise an amino acid sequence comprising a hypervariable region comprising an exogenous targeting peptide having the sequence: optional N-terminal linker -Y-X′-X″-GNPA-X′″-RYFD-X″″, wherein X′ is G, A, R, K, X″ is Y or H, X″ is T, R or H, and X″″ is V or K (SEQ ID NO: 14)—optional C-terminal linker; and
(b) a vector genome packaged in the AAV capsid, wherein the vector genome comprises a nucleic acid sequence encoding a gene product under control of sequences which direct expression thereof.

2. The rAAV of claim 1, wherein at least one of the optional N-terminal linker and/or the optional C-terminal linker are present and are independently selected from a two amino acids linker to a seven amino acids linker.

3. The rAAV of claim 1, wherein the exogenous targeting peptide of SEQ ID NO: 14 and optionally linker(s) are inserted in the hypervariable region VIII (HVRVIII) or in the hypervariable region IV (HVRIV) at a suitable location of a parental AAV capsid.

4. The rAAV of claim 3, wherein the parental capsid is selected from AAV9, AAV8, AAV7, AAV6, AAV5, AAV4, AAV3, AAV1, AAVhu68, and AAVrh91.

5. The rAAV of claim 1, wherein the exogenous targeting peptide of SEQ ID NO: 14 and optionally linker(s) are inserted in the hypervariable region between amino acids 588 and 589 as determined based on the numbering of VP1 amino acid sequence of SEQ ID NO: 9 (in an AAV9 parental capsid).

6. The rAAV of claim 1, wherein the exogenous targeting peptide comprises: Y-X′-X″-GNPA-X′″-RYFD-X″, wherein X′ is G, A, R, K, X″ is Y or H, X′″ is T, R or H, and X″ is V or K (SEQ ID NO: 14).

7. The rAAV of claim 1, wherein the exogenous targeting peptide comprises is selected from:
(SEQ ID NO: 1)
(a) YGYGNPATRYFDV;
or
(SEQ ID NO: 2)
(b) YAYGNPATRYFDV.

8. The rAAV of claim 1, wherein the exogenous targeting peptide comprises is selected from:
(SEQ ID NO: 3)
(a) YKYGNPATRYFDV;
(SEQ ID NO: 4)
(b) YRYGNPATRYFDV;
(SEQ ID NO: 5)
(c) YGHGNPATRYFDV;
(SEQ ID NO: 6)
(d) YGYGNPARRYFDV;
(SEQ ID NO: 7)
(e) YGYGNPAHRYFDV;
or
(SEQ ID NO: 8)
(f) YGYGNPATRYFDK

9. A composition comprising a stock of the rAAV of claim 1 and one or more of a physiologically compatible carrier, excipient, and/or aqueous suspension base.

10. A recombinant brain cell-targeting peptide, the peptide comprising a core targeting motif of an amino acid sequence of Y-X′-X″-GNPA-X′″-RYFD-X″″, wherein X′ is G, A, R, K, X″ is Y or H, X′″ is T, R or H, and X″″ is V or K (SEQ ID NO: 14), optionally flanked at an amino terminus and/or at a carboxy terminus of SEQ ID NO: 14 by two amino acids to seven amino acids, and optionally the peptide or a peptide with linker(s) are conjugated to a nanoparticle, a second molecule, or a recombinant viral capsid protein.

11. The recombinant brain cell-targeting peptide of claim 10, wherein the peptide comprises: Y-X′-X″-GNPA-X′″-RYFD-X″, wherein X′ is G, A, R, K, X″ is Y or H, X′″ is T, R or H, and X″ is V or K (SEQ ID NO: 14).

12. The recombinant brain cell-targeting peptide of claim 10, wherein the peptide comprises amino acid sequence:
(SEQ ID NO: 1)
(a) YGYGNPATRYFDV;
or
(SEQ ID NO: 2)
(b) YAYGNPATRYFDV

13. The recombinant brain cell-targeting peptide of claim 10, wherein the peptide comprises amino acid sequence:
(SEQ ID NO: 3)
(a) YKYGNPATRYFDV;
(SEQ ID NO: 4)
(b) YRYGNPATRYFDV;
(SEQ ID NO: 5)
(c) YGHGNPATRYFDV;
(SEQ ID NO: 6)
(d) YGYGNPARRYFDV;
(SEQ ID NO: 7)
(e) YGYGNPAHRYFDV;
or
(SEQ ID NO: 8)
(f) YGYGNPATRYFDK.

14. The recombinant brain cell-targeting peptide of claim 10, wherein the amino acid sequence of the core targeting motif is YGYGNPATRYFDV (SEQ ID NO: 1).

15. The recombinant brain cell targeting peptide of claim 10, wherein the amino acid sequence of the core targeting motif is YAYGNPATRYFDV (SEQ ID NO: 2).

16. A nucleic acid molecule encoding the recombinant brain cell-targeting peptide of claim 10.

17. A composition comprising the recombinant brain cell-targeting peptide of claim 10 and one or more of a physiologically compatible carrier, excipient, and/or aqueous suspension base.

18. A fusion polypeptide or protein comprising the recombinant brain cell-targeting peptide of claim 10 and a fusion partner which comprises at least one polypeptide or protein.

19. A composition comprising a fusion polypeptide or protein of claim 18 and one or more of a physiologically compatible carrier, excipient, and/or aqueous suspension base.

20. (canceled)

21. A method for targeted therapy to brain cells comprising administering to a patient in need thereof a stock of the rAAV of claim 1, wherein the encoded gene product a therapeutic is targeted for delivery to cells in brain and/or spinal cord.

22. The method for targeted therapy to brain cells of claim 21, wherein the encoded gene product is targeted for delivery to cells in brain and/or spinal cord, and is de-targeted for cells in liver, heart and/or lung.

23. A method for treating Allan-Herndon-Dudley disease by delivering to a subject in need thereof a stock of the rAAV of claim 1, wherein the encoded gene product is an MCT8 protein.

24. (canceled)

25. A method for treating of one or more of cognitive disorders, neurodegenerative disorders, various infections of the central nervous system, and/or a disease of the central nervous system by delivering to a subject in need thereof a stock of the rAAV of claim 1, wherein the encoded gene product is a protein, or an antibody.

26. (canceled)

27. A method for increasing transduction of AAV production cells in vitro comprising transducing cells with a nucleic acid sequence encoding an AAV capsid comprising an exogenous targeting peptide of Y-X′-X″-GNPA-X′″-RYFD-X″″ core motif, wherein X′ is G, A, R, K, X″ is Y or H, X″ is T, R or H, and X″″ is V or K (SEQ ID NO: 14).

28. The method of claim 27, wherein the production cells are 293 cells.


SEQUENCE LISTING (65 sequences)

SEQ ID NO: 1 - protein, 13 aa, Artificial Sequence
  YGY peptide sequence
YGYGNPATRYFDV

SEQ ID NO: 2 - protein, 13 aa, Artificial Sequence
  YGY2A peptide sequence
YAYGNPATRYFDV

SEQ ID NO: 3 - protein, 13 aa, Artificial Sequence
  YGY2K peptide sequence
YKYGNPATRYFDV

SEQ ID NO: 4 - protein, 13 aa, Artificial Sequence
  YGY2R peptide sequence
YRYGNPATRYFDV

SEQ ID NO: 5 - protein, 13 aa, Artificial Sequence
  YGY3H peptide sequence
YGHGNPATRYFDV

SEQ ID NO: 6 - protein, 13 aa, Artificial Sequence
  YGY8R peptide sequence
YGYGNPARRYFDV

SEQ ID NO: 7 - protein, 13 aa, Artificial Sequence
  YGY8H peptide sequence
YGYGNPAHRYFDV

SEQ ID NO: 8 - protein, 13 aa, Artificial Sequence
  YGY13K peptide sequence
YGYGNPATRYFDK

SEQ ID NO: 9 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 7 aa, Artificial Sequence
  PHP.B peptide sequence
TLAVPFK

SEQ ID NO: 12 - protein, 7 aa, Artificial Sequence
  AAV2 variant peptide sequence
NDVRAVS

SEQ ID NO: 13 - protein, 621 aa, adeno-associated virus 2
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 14 - protein, 13 aa, Artificial Sequence
  Y-G/A/R/K-Y/H-GNPA-T/R/H-RYFD-V/K motif
  MISC_FEATURE (2)..(2): Xaa is selected from Glycine (G), Alanine (A),  Arginine (R), or Lysine (K)
  MISC_FEATURE (3)..(3): Xaa is selected from Tyrosine (Y), or Histidine (H)
  MISC_FEATURE (8)..(8): Xaa is selected from Threonine (T), Arginine (R), or Histidine (H)
  MISC_FEATURE (13)..(13): Xaa is selected from Valine (V), or Lysine (K)
YXXGNPAXRYFDX

SEQ ID NO: 15 - protein, 10 aa, Artificial Sequence
  HYL peptide
HYLGYAWVGG

SEQ ID NO: 16 - protein, 11 aa, Artificial Sequence
  EFS peptide
EFSSNTVKLTS

SEQ ID NO: 17 - protein, 10 aa, Artificial Sequence
  SAN peptide
SANFIKPTSY

SEQ ID NO: 18 - DNA, 4731 bp, Artificial Sequence
  AAV2/9 n588 YGY2A nucleic acid sequence expression cassette
  misc_feature (1)..(36): truncated promoter
  misc_feature (1)..(7): p5 promoter
  CDS (37)..(1902): AAV2 rep
  CDS (1919)..(4168): AAV9-YGY2A Cap
  misc_feature (3683)..(3721): YGY2A
  misc_feature (4258)..(4389): P5 promoter
  misc_feature (4437)..(4731): LacZ promoter
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatatgcgtatggcaacccggcgacccgttattttgatgtggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 19 - protein, 621 aa, Artificial Sequence; identical to SEQ ID NO: 13
  Synthetic Construct

SEQ ID NO: 20 - protein, 749 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQYAYGNPATRYFDVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 21 - DNA, 4731 bp, Artificial Sequence
  AAV2/9 n588 YGY nucleic acid sequence expression cassette
  misc_feature (1)..(36): truncated promoter
  misc_feature (1)..(7): p5 promoter
  CDS (37)..(1902): AAV2 rep
  CDS (1919)..(4168): AAV9-YGY cap
  misc_feature (3683)..(3721): YGY
  misc_feature (4259)..(4389): P5 promoter
  misc_feature (4517)..(4731): LacZ promoter
ccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatacggctacggcaaccccgccacccgctacttcgacgtggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 22 - protein, 621 aa, Artificial Sequence; identical to SEQ ID NO: 13
  Synthetic Construct

SEQ ID NO: 23 - protein, 749 aa, Artificial Sequence
  Synthetic Construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQYGYGNPATRYFDVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - DNA, 39 bp, Artificial Sequence
  nucleic acid sequence YGY2A
tatgcgtatggcaacccggcgacccgttattttgatgtg

SEQ ID NO: 25 - DNA, 39 bp, Artificial Sequence
  nucleic acid sequence YGY
tacggctacggcaaccccgccacccgctacttcgacgtg

SEQ ID NO: 26 - DNA, 2250 bp, Artificial Sequence
  AAV9 cap n588 YGY2A nucleic acid sequence
  misc_feature (1765)..(1803): YGY2A
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatatgcgtatggcaacccggcgacccgttattttgatgtggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 27 - protein, 749 aa, Artificial Sequence; identical to SEQ ID NO: 20
  AAV9 cap n588 YGY2A amino acid sequence
  MISC_FEATURE (589)..(601): YGY2A

SEQ ID NO: 28 - DNA, 2250 bp, Artificial Sequence
  AAV9 cap n588 YGY nucleic acid sequence
  misc_feature (1765)..(1803): YGY
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaatacggctacggcaaccccgccacccgctacttcgacgtggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 29 - protein, 749 aa, Artificial Sequence; identical to SEQ ID NO: 23
  AAV9 cap n588 YGY amino acid sequence
  MISC_FEATURE (589)..(601): YGY

SEQ ID NO: 30 - protein, 50 aa, Artificial Sequence
  amino acids 566 to 615 of adeno-associated virus 9 capsid
IKTTNPVATESYGQVATNHQSAQAQAQTGWVQNQGILPGMVWQDRDVYLQ

SEQ ID NO: 31 - protein, 50 aa, Artificial Sequence
  amino acids 565 to 614 of adeno-associated virus 8 capsid
EEEIKTTNPVATEEYGIVADNLQQQNTAPQIGTVNSQGALPGMVWQNRDV

SEQ ID NO: 32 - protein, 50 aa, Artificial Sequence
  amino acids 567 to 616 of adeno-associated virus 7 capsid
IRPTNPVATEEYGIVSSNLQAANTAAQTQVVNNQGALPGMVWQNRDVYLQ

SEQ ID NO: 33 - protein, 50 aa, Artificial Sequence
  amino acids 550 to 599 of adeno-associated virus 6 capsid
ASNTALDNVMITDEEEIKATNPVATERFGTVAVNLQSSSTDPATGDVHVM

SEQ ID NO: 34 - protein, 50 aa, Artificial Sequence
  amino acids 556 to 605 of adeno-associated virus 5 capsid
QPVNRVAYNVGGQMATNNQSSTTAPATGTYNLQEIVPGSVWMERDVYLQG

SEQ ID NO: 35 - protein, 50 aa, Artificial Sequence
  amino acids 558 to 607 of adeno-associated virus 4 capsid
FTSEEELAATNATDTDMWGNLPGGDQSNSNLPTVDRLTALGAVPGMVWQN

SEQ ID NO: 36 - protein, 50 aa, Artificial Sequence
  amino acids 564 to 613 of adeno-associated virus 3B capsid
EEIRTTNPVATEQYGTVANNLQSSNTAPTTRTVNDQGALPGMVWQDRDVY

SEQ ID NO: 37 - protein, 50 aa, Artificial Sequence
  amino acids 566 to 615 of adeno-associated virus 2 capsid
RTTNPVATEQYGSVSTNLQRGNRQAATADVNTQGVLPGMVWGDRDVYLQG

SEQ ID NO: 38 - protein, 50 aa, Artificial Sequence
  amino acids 566 to 615 of adeno-associated virus 1 capsid
IKATNPVATERFGTVAVNFQSSSTDPATGDVHAMGALPGMVWQDRDVYLQ

SEQ ID NO: 39 - DNA, 542 bp, Artificial Sequence
  WPRE element (mut)
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 40 - DNA, 591 bp, Artificial Sequence
  Wild Type WPRE element (GenBank J02442.1, nt 1093-1683)
aatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcct

SEQ ID NO: 41 - protein, 11 aa, Artificial Sequence
  SSNTVKLTSGH peptide sequence
SSNTVKLTSGH

SEQ ID NO: 42 - protein, 15 aa, Artificial Sequence
  GRTILKENIKYEVAI peptide sequence
GRTILKENIKYEVAI

SEQ ID NO: 43 - protein, 19 aa, Artificial Sequence
  GGGRTILKENIKYEVAIGG peptide sequence
GGGRTILKENIKYEVAIGG

SEQ ID NO: 44 - protein, 15 aa, Artificial Sequence
  GGSSNTVKLTSGHGG peptide sequence
GGSSNTVKLTSGHGG

SEQ ID NO: 45 - protein, 12 aa, Artificial Sequence
  IEINATRAGTNL peptide sequence
IEINATRAGTNL

SEQ ID NO: 46 - protein, 16 aa, Artificial Sequence
  GGVLTNIARGEYMRGG peptide sequence
GGVLTNIARGEYMRGG

SEQ ID NO: 47 - protein, 16 aa, Artificial Sequence
  GGIEINATRAGTNLGG peptide sequence
GGIEINATRAGTNLGG

SEQ ID NO: 48 - protein, 19 aa, Artificial Sequence
  GGPVEFSSNTVKLTSGHGG peptide sequence
GGPVEFSSNTVKLTSGHGG

SEQ ID NO: 49 - protein, 15 aa, Artificial Sequence
  GGFLGTPADTGHGGG peptide sequence
GGFLGTPADTGHGGG

SEQ ID NO: 50 - protein, 12 aa, Artificial Sequence
  LHRQDNYLAASG peptide sequence
LHRQDNYLAASG

SEQ ID NO: 51 - protein, 15 aa, Artificial Sequence
  GGFTSVGKAVHQVGG peptide sequence
GGFTSVGKAVHQVGG

SEQ ID NO: 52 - protein, 7 aa, Artificial Sequence
  PMKLDSS peptide sequence
PMKLDSS

SEQ ID NO: 53 - protein, 7 aa, Artificial Sequence
  ISQTLHG peptide sequence
ISQTLHG

SEQ ID NO: 54 - protein, 13 aa, Artificial Sequence
  GSGLHGPSSTGSG peptide sequence
GSGLHGPSSTGSG

SEQ ID NO: 55 - protein, 13 aa, Artificial Sequence
  GSGISQTLHGGSG peptide sequence
GSGISQTLHGGSG

SEQ ID NO: 56 - protein, 13 aa, Artificial Sequence
  GSGHTLHGSAGSG peptide sequence
GSGHTLHGSAGSG

SEQ ID NO: 57 - protein, 11 aa, Artificial Sequence
  GSVGGVFTSVG peptide sequence
GSVGGVFTSVG

SEQ ID NO: 58 - protein, 7 aa, Artificial Sequence
  STSNLAS peptide sequence
STSNLAS

SEQ ID NO: 59 - protein, 7 aa, Artificial Sequence
  STASTQA peptide sequence
STASTQA

SEQ ID NO: 60 - protein, 15 aa, Artificial Sequence
  DFGSVGGVFTSVGKA peptide sequence
DFGSVGGVFTSVGKA

SEQ ID NO: 61 - protein, 7 aa, Artificial Sequence
  RASNLES peptide sequence
RASNLES

SEQ ID NO: 62 - protein, 13 aa, Artificial Sequence
  GSGMLVSSPAGSG peptide sequence
GSGMLVSSPAGSG

SEQ ID NO: 63 - protein, 19 aa, Artificial Sequence
  GGDFGSVGGVFTSVGKAGG peptide sequence
GGDFGSVGGVFTSVGKAGG

SEQ ID NO: 64 - protein, 7 aa, Artificial Sequence
  SYTSSTM peptide sequence
SYTSSTM

SEQ ID NO: 65 - protein, 7 aa, Artificial Sequence
  HTLHGSA peptide sequence
HTLHGSA